(19) United States
(12) Patent Application Publication
(10) Pub. No.: US 2023/0304994 A1
(43) Pub. Date: Sep. 28, 2023

(54) METHODS AND ASSAYS FOR ANALYZING SECRETOME-CONTAINING COMPOSITIONS

(71) Applicant: FUJIFILM Corporation, Tokyo (JP)

(72) Inventors: Nisa K. E. RENAULT, Madison, WI (US); Michele L. HAMRICK, Madison, WI (US); Coby Carlson, Madison, WI (US); Megan Livingston, Madison, WI (US)

(73) Assignee: FUJIFILM Corporation, Tokyo (JP)

(21) Appl. No.: 18/319,265

(22) Filed: May 17, 2023

Related U.S. Application Data
(63) Continuation of application No. PCT/IB2021/000794, filed on Nov. 17, 2021.
(60) Provisional application No. 63/115,242, filed on Nov. 18, 2020.

(51) Int. Cl. G01N 33/50; C12N 5/077; C12N 5/00

(52) U.S. Cl. CPC G01N 33/5044; C12N 5/0657; C12N 5/0018; C12N 2500/30

(57) ABSTRACT

The present disclosure provides methods for generating and/or purifyingsecretomes, extracellular vesicles, and fractions thereof, from cells, such as progenitor cells; and methods for analyzing activities, and the functionality and potency, of such secretomes, extracellular vesicles, and fractions thereof. The present disclosure also relates to the therapeutic use of secretomes, extracellular vesicles, and fractions thereof, analyzed using such methods.

### CROSS-REFERENCE TO RELATED APPLICATIONS

This application is a Continuation of PCT International Application No. PCT/IB2021/000794 filed on Nov. 17, 2021, which claims priority under 35 U.S.C. § 119 to U.S. Provisional Patent Application No. 63/115,242 filed on Nov. 18, 2020. Each of the above application(s) is hereby expressly incorporated by reference, it its entirety, into the present application.

### FIELD OF THE INVENTION

The present disclosure relates generally to methods and assays for analyzing an activity, functionality, characteristic, and/or potency, of conditioned media; or of a

secretome-, extracellular vesicle-, and/or a small extracellular vesicle-enriched fraction (sEV)-containing composition.

### BACKGROUND INFORMATION

Cells, including those in in vitro or ex vivo culture, secrete a large variety of molecules and biological factors (collectively known as a secretome) into the extracellular space. See Vlassov et al. (Biochim Biophys Acta, 2012; 940-948). As part of the secretome, various bioactive molecules are secreted from cells within membrane-bound extracellular vesicles, such as exosomes. Extracellular vesicles are capable of altering the biology of other cells through signaling, or by the delivery of their cargo (including, for example, proteins, lipids, and nucleic acids). The cargo of extracellular vesicles is encased in a membrane which, amongst others, allows for specific targeting (e.g., to target cells) via specific markers on the membrane; and increased stability during transport in biological fluids, such as through the bloodstream or across the blood-brain-barrier (BBB).

Exosomes exert a broad array of important physiological functions, e.g., by acting as molecular messengers that traffic information between different cell types. For example, exosomes deliver proteins, lipids and soluble factors including RNA and microRNAs which, depending on their source, participate in signaling pathways that can influence apoptosis, metastasis, angiogenesis, tumor progression, thrombosis, immunity by directing T cells towards immune activation, immune suppression, growth, division, survival, differentiation, stress responses, apoptosis, and the like. See Vlassov et al. (Biochim Biophys Acta, 2012; 940-948). Extracellular vesicles may contain a combination of molecules that may act in concert to exert particular biological effects. Exosomes incorporate a wide range of cytosolic and membrane components that reflect the properties of the parent cell. Therefore, the terminology applied to the originating cell can in some instances be used as a simple reference for the secreted exosomes.

Progenitor cells have proliferative capacity and can differentiate into mature cells, making progenitor cells attractive for therapeutic applications such as regenerative medicine, e.g., in treating myocardial infarction and congestive heart failure. It has been reported that extracellular vesicles secreted by stem cell-derived cardiovascular progenitor cells produce similar therapeutic effects to their secreting cells in a mouse model of chronic heart failure, see Kervadec et al. (J. Heart Lung Transplant, 2016; 35:795-807), suggesting that a significant mechanism of action of transplanted progenitor cells is in the release of biological factors following transplantation (e.g., which stimulate endogenous regeneration or repair pathways). This raises the possibility of effective, cell-free therapies (with benefits such as improved convenience, stability, and operator handling). See El Harane et al. (Eur. Heart J., 2018; 39:1835-1847). However, there currently is a need for more accurate and reliable methods for determining an activity, functionality, or potency, of conditioned media; or of a secretome-, extracellular vesicle-, and/or a sEV-containing composition.

For conditioned media, or a secretome-, extracellular vesicle-, and/or sEV-containing composition, a known cell viability assay involving serum-deprivation can be used to determine the functionality or potency thereof. In particular, a well-known cardiomyocyte viability assay uses serum-deprived rat H9c2 cardiomyoblasts, and may be used to measure the effect of a conditioned media, or a secretome-, extracellular vesicle-, and/or sEV-containing composition, on the viability of the serum-deprived cells. See, e.g., El Harane et al. (Eur. Heart J., 2018, 39(20): 1835-1847).

However, H9c2 cardiomyoblasts, which are ubiquitously used in such an assay, have been reported to exhibit variation in cell parameters following repeated passage. For instance, Witek et al. (Cytotechnology, 2016, 68(6): 2407-2415) reported that cell parameters (such as cell morphology, gene expression profile, oxidative stress response, and sensitivity to cellular stressors) varied according to the age of the cell culture (which could lead to unreliable results when, e.g., testing agents for their effect on promoting or decreasing cell viability). Additionally, few options exist for the screening of secretomes (e.g., from different cell types and/or different cell culture conditions) for their therapeutic potential.

Accordingly, there remains a need for improved and more reliable assays for determining an activity, functionality and/or potency of conditioned media; or of secretome-, extracellular vesicle-, and/or sEV-containing compositions.

### SUMMARY OF THE INVENTION

The present disclosure addresses the above-described limitations in the art, by providing methods and assays for reliably determining an activity, functionality and/or potency of conditioned media, secretomes, extracellular vesicles, and fractions thereof. The present disclosure further provides reliable analysis methods and assays for comparing conditioned media, secretomes, extracellular vesicles, and fractions thereof, allowing high-throughput analyses, and capable of producing consistent results in vitro.

Non-limiting embodiments of the disclosure include as follows:
[1] A method for analyzing an activity of a secretome, said method comprising: (a) contacting a culture of target cells with a pre-treatment medium, and culturing said target cells in said pre-treatment medium under at least one stress-inducing condition; (b) administering a secretome to the cell culture, and culturing said target cells in the presence of the secretome; and (c) measuring at least one property of the cultured cells one or more times during the culturing of step (b).
[2] The method of [1], wherein said method further comprises removing the pre-treatment medium from the cultured cells before step (b).
[3] The method of [2], wherein the target cells are cultured under the at least one stress-inducing condition before the administration of the secretome to the cell culture, and wherein the culturing of the target cells in step (b) is performed in the absence of the at least one stress-inducing condition.
[4] A method for analyzing an activity of a secretome, said method comprising: (a) contacting a culture of target cells with a pre-treatment medium, and culturing said target cells in said pre-treatment medium; (b) administering a secretome to the cell culture, and optionally culturing said target cells in the presence of the secretome; (c) culturing the target cells under at least one stress-inducing condition; and (d) measuring at least one property of the cultured cells one or more times during the culturing of step (c).
[5] The method of [4], wherein said target cells are cultured in the presence of the secretome before the culturing of step (c).
[6] The method of [5], wherein said method further comprises removing the secretome from the cultured cells before step (c).
[7] The method of [4], wherein the stress-inducing condition is culturing in the presence of a cellular stress agent, wherein the cellular stress agent is co-administered with the secretome, and wherein said target cells are cultured in the presence of the secretome and the cellular stress agent.
[8] The method of any one of [1]-[6], wherein the at least one stress-inducing culture condition is culturing in the presence of a cellular stress agent.
[9] The method of [7] or [8], wherein said cellular stress agent is a chemotherapeutic agent and/or an apoptosis-inducing agent.
[10] The method of [9], wherein said apoptosis-inducing agent is an indolocarbazole.
[11] The method of [9], wherein said apoptosis-inducing agent is an indolo(2,3-a)pyrrole(3,4-c)carbazole or a derivative thereof.
[12] The method of [9], wherein said apoptosis-inducing agent is staurosporine, or a derivative thereof.
[13] The method of [9], wherein said apoptosis-inducing agent is doxorubicin, or a derivative thereof.
[14] The method of any one of [1]-[13], wherein the at least one property that is measured is selected from the group consisting of cell viability, hypertrophy, cell health, cell adhesion, cell physiology, ATP content, cell number, and cell morphology.
[15] The method of [14], wherein said method further comprises measuring the at least one property of the cultured cells one or more times during the culturing of step (a).
[16] The method of [1]-[3], wherein the at least one property is measured multiple times during the culturing of step (b).
[17] The method of [4]-[7], wherein the at least one property is measured multiple times during the culturing of step (c).
[18] The method of [16] or [17], wherein the multiple measurements are made 5 minutes to 10 hours apart from each other.
[19] The method of [18], wherein the multiple measurements are made 10 minutes to 4 hours apart from each other.
[20] The method of [19], wherein the multiple measurements are made 30 minutes to 2 hours apart from each other.
[21] The method of any one of [1]-[20], wherein the at least one property is selected from cell viability, cell adhesion, cell number, cell morphology, cell growth, and/or ATP content.
[22] The method of [21], wherein the at least one property is viability of the cultured cells, and the viability is measured using a fluorescent DNA-labeling dye or a fluorescent nuclear-staining dye.
[23] The method of [21], wherein the at least one property is adhesion, cell number, growth, and/or morphology of the cultured cells, and wherein the adhesion, cell number, growth, and/or morphology of the cultured cells is determined by measuring electrical impedance across a culture vessel surface in the culture.
[24] The method of any one of [1]-[23], wherein the culturing of said target cells in said pre-treatment medium in step (a) is for 30 minutes to 10 hours.
[25] The method of [24], wherein the culturing of said target cells in said pre-treatment medium in step (a) is for 1 hour to 5 hours.
[26] The method of [25], wherein the culturing of said target cells in said pre-treatment medium in step (a) is for about 4 hours.
[27] The method of any one of [1]-[3], wherein the culturing of said target cells in step (b) is for at least 2 hours.
[28] The method of any one of [4]-[7], wherein the culturing of said target cells in step (c) is for at least 2 hours.
[29] The method of [27] or [28], wherein the culturing is for at least 5 hours.
[30] The method of [29], wherein the culturing is for at least 12 hours.
[31] The method of [30], wherein the culturing is for at least 24 hours.
[32] The method of [22], wherein the viability of the cultured cells is measured by imaging the DNA-labeling dye or the nuclear-staining dye.
[33] The method of any one of [1]-[20] and [24]-[31], wherein the at least one property is viability of the cultured cells, and the viability is measured by live cell imaging.
[34] The method of any one of [1]-[33], wherein said target cells are specialized cells.
[35] The method of any one of [1]-[33], wherein said target cells comprise cardiomyocytes, cardiovascular progenitors, cardiac progenitors, and/or vascular cells.
[36] The method of [34], wherein said specialized cells are obtained from induced pluripotent stem cells (iPSCs).
[37] The method of any one of [1]-[33], wherein said target cells have previously been frozen.
[38] The method of any one of [1]-[37], wherein said secretome is isolated from a culture of one or more cells selected from totipotent progenitor cells, multipotent progenitor cells, and end-stage differentiated cells.
[39] The method of [38], wherein said one or more progenitor cells comprise progenitor cells selected from the group consisting of cardiomyocyte progenitor cells, cardiac progenitor cells, cardiovascular progenitor cells, and mesenchymal stem cells.
[40] The method of any one of [1]-[39], wherein said secretome comprises a small extracellular vesicle-enriched fraction (sEV) isolated from a cell culture.
[41] The method of [40], wherein said sEV has one or more of the following characteristics: (a) is CD63⁺, CD81⁺ and/or CD9⁺; (b) comprises extracellular vesicles that are between 50-200 nm in diameter; (c) are positive for one or more of CD49e, ROR1 (Receptor Tyrosine Kinase Like Orphan Receptor 1), SSEA-4 (Stage-specific embryonic antigen 4), MSCP (Mesenchymal stem cell-like protein), CD146, CD41b, CD24, CD44, CD236, CD133/1, CD29 and CD142; and/or (d) is negative for one or more of CD19, CD4, CD209, HLA-ABC (human leukocyte antigen-ABC), CD62P, CD42a and CD69.
[42] The method of [40], wherein said sEV comprises one or more of exosomes, microparticles, extracellular vesicles and secreted proteins.
[43] The method of any one of [1]-[42], wherein said method further comprises culturing the target cells for 1-21 days prior to step (a).
[44] The method of [43], wherein the target cells have been cultured for 5-14 days prior to step (a).
[45] The method of [43], wherein the target cells are supplied with fresh culture medium 12-36 hours prior to step (a).
[46] The method of any one of [1]-[45], wherein the target cells are cultured in two-dimensional cell culture.
[47] The method of [46], wherein said two-dimensional cell culture comprises culturing the target cells on a culture vessel surface.
[48] The method of [47], wherein said culture vessel surface is coated with a substance to promote cell adhesion.
[49] The method of [48], wherein said substance to promote cell adhesion is fibronectin.
[50] The method of any one of [1]-[49], wherein said method further comprises culturing positive control cells in parallel, wherein said positive control cells are not administered the secretome, and are not subjected to the stress-inducing condition.
[51] The method of any one of [1]-[50], wherein said method further comprises culturing negative control cells, wherein said negative control cells are not administered the secretome.
[52] The method of [51], wherein said negative control cells comprise negative control cells subjected to the same steps as the target cells, except that they are not administered the secretome.
[53] The method of [51] or [52], wherein said negative control cells comprise negative control cells cultured in the pre-treatment medium under the at least one stress-inducing condition, and said method comprises measuring the at least one property of the negative control cells during or after the culturing in the pre-treatment medium under the at least one stress-inducing condition.
[54] The method of [51], wherein said negative control cells comprise negative control cells to which a mock secretome composition is added, wherein said mock secretome composition is produced by omitting cells from the process of producing a secretome.
[55] The method of any one of [1]-[54], wherein the amount of secretome added to said target cells is determined based on one or more of: the amount of secreting cells that produced the secretome; the protein content of said secretome; the RNA content of said secretome; the exosome amount of said secretome; and the number of particles in said secretome.
[56] The method of [50], wherein said target cells and said positive control cells are cultured in replicate.
[57] The method of [56], wherein the number of positive control cells in the replicate cultures are averaged to produce an average maximum cell number, and the number of target cells in each replicate culture is normalized to the average maximum cell number.
[58] The method of any one of [51]-[54], wherein the at least one property is viability of the cultured cells, and wherein said secretome is determined to have potency and/or exhibit a therapeutic effect when the viability of the target cells is higher than the viability of the negative control cells.
[59] The method of [40], wherein said sEV is at least one of the following: a sEV that has been enriched for extracellular vesicles having a diameter of between about 50-200 nm or between 50-200 nm, preferably having a diameter of between about 50-150 nm or between 50-150 nm; a sEV that is substantially free or free of whole cells; and/or a sEV that is substantially free of one or more culture medium components.
[60] A method for analyzing an activity of a small molecule or chemotherapy agent, said method comprising: (a) contacting a culture of target cells with a pre-treatment medium, and culturing said target cells in said pre-treatment medium in the presence of the small molecule or chemotherapy agent; (b) administering a secretome to the cell culture, and culturing said target cells in the presence of the secretome; and (c) measuring at least one property of the cultured cells one or more times during the culturing of step (b).

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts representative images for cells incubated with and without staurosporine in a staurosporine cardiomyocyte viability assay. Nuclei in healthy cells appear round and of medium red intensity (as found in panel 1), whereas dead and dying nuclei appear shriveled and intensely red (as found in panel 3). The Incucyte image analysis software identifies viable nuclei (blue “masking” in panels 2 and 4) and counts them in each image.

FIG. 2 depicts a time course measuring cell viability in a staurosporine cardiomyocyte viability assay. Following staurosporine pre-treatment, cell viability was measured each hour after administration of either: small extracellular vesicle enriched fraction (sEV) secretomes obtained from human cardiovascular progenitor cells (CPC); mock sEV preparations (Virgin media controls); or complete media without sEVs. The complete media control (positive control) was not subjected to the staurosporine pre-treatment.

FIG. 3 depicts a histogram showing the results at the 24-hour time point of a staurosporine cardiomyocyte viability assay time course. The values for such histograms are calculated first by normalizing the viable cell count at 24-hrs to the viable cell count at 0-hrs for the same well. Next, the normalized baseline result (as determined in the negative control wells), is subtracted from each result. Finally, the results of each condition are represented as a percentage of the average of the positive control wells. Averages and standard deviations for each condition are shown.

FIG. 4 depicts, for a staurosporine assay as described in Example 2 using sEVs produced from CPCs, a histogram showing cell viability data (obtained using biocompatible dyes, measured using an Incucyte at the 13 h timepoint), as well as ATP quantification data (obtained using the CellTiter-Glo® Luminescent Cell Viability Assay (Promega), measured with CLARIOStar® (BMG Labtech) and Tecan for Life Science® plate readers (at the 24 h timepoint, i.e., after the Incucyte time course had ended)).

FIG. 5 depicts, for a staurosporine assay as described in Example 2 using sEVs produced from MSCs, histograms showing cell viability data (obtained using biocompatible dyes, measured using an Incucyte at the 12 h timepoint), as well as ATP quantity data (obtained using the CellTiter-Glo® Luminescent Cell Viability Assay (Promega) measured with CLARIOStar® (BMG Labtech) and Tecan for Life Science® plate readers (at the 23 h timepoint, i.e., after the Incucyte time course had ended)).

FIG. 6 depicts, for a staurosporine assay as described in Example 2 using sEVs produced from MSCs, histograms showing ATP quantity data (obtained using the CellTiter-Glo® Luminescent Cell Viability Assay (Promega) measured with a Tecan for Life Science® plate reader (at the 23 h timepoint). The sEV treatment was conducted either in serum containing medium (“iCell Cardiomyocyte Maintenance Medium M1003”), or in serum free medium (“iCell Cardiomyocyte Serum-Free Medium M1038”), as depicted.

FIG. 7 depicts a comparison between the results of a staurosporine cardiomyocyte viability assay and a HUVEC scratch wound healing assay, which determined the effects of sEV preparations on angiogenesis. Similar trends in efficacy were found for the samples tested in parallel in these two assays, indicating that for cardiovascular progenitor cell-sEV, there are effects on both cardiac and vascular target cells in vitro.

FIG. 8 depicts the results of a scratch wound healing assay, showing that sEV isolated from MSC from three different donors (62, 64, 82) appear to have vastly different effects on wound healing when dosed based on particle number (left graphs). However, when dosed based on secreting cell number (right graph), the sEV from different MSC donors appear to have far more similar effects in this assay.

FIG. 9 depicts a time course measuring electrical impedance, in a staurosporine cardiomyocyte cell adhesion/number/growth assay. Following pre-treatment (with or without staurosporine), cell adhesion/number/growth was continually analyzed (by measuring electrical impedance) before and after administration of either: sEV obtained from human cardiovascular progenitor cells (“sEV”, Conditions 3-5); mock sEV preparations (virgin media controls (“MV”; Conditions 1 and 7)); or media/culture controls without sEVs (Conditions 2, 6 and 8).

FIG. 10 shows the results of the time course experiment depicted in FIG. 9, normalized to the treatment (“Tx”) timepoint.

### DETAILED DESCRIPTION OF THE INVENTION

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a cell” includes one or more cells.

Unless defined otherwise, all technical and scientific terms used herein have the same meaning as commonly understood by one of ordinary skill in the art to which the invention pertains. Although other methods and materials similar, or equivalent, to those described herein can be useful in the present invention, preferred materials and methods are described herein.

As used herein, “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals as well as males and females. In some embodiments, cells (for example, stem cells, including pluripotent stem cells, progenitor cells, or tissue-specific cells) are derived from a subject. In some embodiments, the subject is a non-human subject.

As used herein, “differentiation” refers to processes by which unspecialized cells (such as pluripotent stem cells, or other stem cells), or multipotent or oligopotent cells, for example, acquire specialized structural and/or functional features characteristic of more mature, or fully mature, cells. “Transdifferentiation” is a process of transforming one differentiated cell type into another differentiated cell type.

As used herein, “embryoid bodies” refers to three-dimensional aggregates of pluripotent stem cells. These cells can undergo differentiation into cells of the three germ layers, the endoderm, mesoderm and ectoderm. The three-dimensional structure, including the establishment of complex cell adhesions and paracrine signaling within the embryoid body microenvironment, enables differentiation and morphogenesis.

As used herein, “stem cell” refers to a cell that has the capacity for self-renewal, i.e., the ability to go through numerous cycles of cell division while maintaining their non-terminally-differentiated state. Stem cells can be totipotent, pluripotent, multipotent, oligopotent, or unipotent. Stem cells may be, for example, embryonic, fetal, amniotic, adult, or induced pluripotent stem cells.

As used herein, “pluripotent stem cell” (PSC) refers to a cell that has the ability to reproduce itself indefinitely, and to differentiate into any other cell type of an adult organism. Generally, pluripotent stem cells are stem cells that are capable of inducing teratomas when transplanted in immunodeficient (SCID) mice; are capable of differentiating into cell types of all three germ layers (e.g., can differentiate into ectodermal, mesodermal, and endodermal, cell types); and express one or more markers characteristic of PSCs. Examples of such markers expressed by PSCs, such as embryonic stem cells (ESCs) and iPSCs, include Oct 4, alkaline phosphatase, SSEA-3 surface antigen, SSEA-4 surface antigen, nanog, TRA-1-60, TRA-1-81, SOX2, and REX1.

As used herein, “induced pluripotent stem cell” (iPSC) refers to a type of pluripotent stem cell that is artificially derived from a non-pluripotent cell, typically a somatic cell. In some embodiments, the somatic cell is a human somatic cell. Examples of somatic cells include, but are not limited to, dermal fibroblasts, bone marrow-derived mesenchymal cells, cardiac muscle cells, keratinocytes, liver cells, stomach cells, neural stem cells, lung cells, kidney cells, spleen cells, and pancreatic cells. Additional examples of somatic cells include cells of the immune system, including, but not limited to, B-cells, dendritic cells, granulocytes, innate lymphoid cells, megakaryocytes, monocytes/macrophages, myeloid-derived suppressor cells, natural killer (NK) cells, T cells, thymocytes, and hematopoietic stem cells.

iPSCs may be generated by reprogramming a somatic cell, by expressing or inducing expression of one or a combination of factors (herein referred to as reprogramming factors) in the somatic cell. iPSCs can be generated using fetal, postnatal, newborn, juvenile, or adult somatic cells. In some instances, factors that can be used to reprogram somatic cells to pluripotent stem cells include, for example, OCT4 (OCT3/4), SOX2, c-MYC, and KLF4, NANOG, and LIN28. In some instances, somatic cells may be reprogrammed by expressing at least two reprogramming factors, at least three reprogramming factors, or at least four reprogramming factors, to reprogram a somatic cell to a pluripotent stem cell. The cells may be reprogrammed by introducing reprogramming factors using vectors, including, for example, lentivirus, retrovirus, adenovirus, and Sendai virus vectors. Alternatively, non-viral techniques for introducing reprogramming factors include, for example, mRNA transfection, miRNA infection/transfection, PiggyBac, minicircle vectors, and episomal plasmids. iPSCs may also be generated by, for example, using CRISPR-Cas9-based techniques, to introduce reprogramming factors, or to activate endogenous programming genes.

As used herein, “embryonic stem cells” are embryonic cells derived from embryo tissue, preferably the inner cell mass of blastocysts or morulae, optionally that have been serially passaged as cell lines. The term includes cells isolated from one or more blastomeres of an embryo, preferably without destroying the remainder of the embryo. The term also includes cells produced by somatic cell nuclear transfer. ESCs can be produced or derived from a zygote, blastomere, or blastocyst-staged mammalian embryo produced by the fusion of a sperm and egg cell, nuclear transfer, or parthenogenesis, for example. Human ESCs include, without limitation, MAO1, MAO9, ACT-4, No. 3, H1, H7, H9, H14 and ACT30 embryonic stem cells. Exemplary pluripotent stem cells include embryonic stem cells derived from the inner cell mass (ICM) of blastocyst stage embryos, as well as embryonic stem cells derived from one or more blastomeres of a cleavage stage or morula stage embryo. These embryonic stem cells can be generated from embryonic material produced by fertilization or by asexual means, including somatic cell nuclear transfer (SCNT), parthenogenesis, and androgenesis. PSCs alone cannot develop into a fetal or adult animal when transplanted in utero because they lack the potential to contribute to all extraembryonic tissue (e.g., placenta in vivo or trophoblast in vitro).

As used herein, the term “progenitor cell” refers to a descendant of a stem cell which is capable of further differentiation into one or more kinds of specialized cells, but which cannot divide and reproduce indefinitely. That is, unlike stem cells (which possess an unlimited capacity for self-renewal), progenitor cells possess only a limited capacity for self-renewal. Progenitor cells may be multipotent, oligopotent, or unipotent, and are typically classified according to the types of specialized cells they can differentiate into. For instance, a “cardiomyocyte progenitor cell” is a progenitor cell derived from a stem cell that has the capacity to differentiate into a cardiomyocyte. Similarly, “cardiac progenitor cells” may differentiate into multiple specialized cells constituting cardiac tissue, including, for example, cardiomyocytes, smooth muscle cells, and endothelial cells. Additionally, a “cardiovascular progenitor cell” has the capacity to differentiate into, for example, cells of cardiac and vascular lineages. A mesenchymal stem cell (MSC) is another type of progenitor cell.

As used herein, “expand” or “proliferate” may refer to a process by which the number of cells in a cell culture is increased due to cell division.

“Multipotent” implies that a cell is capable, through its progeny, of giving rise to several different cell types found in an adult animal.

“Pluripotent” implies that a cell is capable, through its progeny, of giving rise to all the cell types that comprise the adult animal, including the germ cells. Embryonic stem cells, induced pluripotent stem cells, and embryonic germ cells are pluripotent cells under this definition.

The term “autologous cells” as used herein refers to donor cells that are genetically identical with the recipient.

As used herein, the term “allogeneic cells” refers to cells derived from a different, genetically non-identical, individual of the same species.

The term “totipotent” as used herein can refer to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps.

As used herein, the term “extracellular vesicles” collectively refers to biological nanoparticles derived from cells, and examples thereof include exosomes, ectosomes, exovesicles, microparticles, microvesicles, nanovesicles, blebbing vesicles, budding vesicles, exosome-like vesicles, matrix vesicles, membrane vesicles, shedding vesicles, membrane particles, shedding microvesicles, oncosomes, exomeres, and apoptotic bodies, but are not limited thereto.

Extracellular vesicles can be categorized, for example, according to size. For instance, as used herein, the term “small extracellular vesicle” refers to extracellular vesicles having a diameter of between about 50-200 nm. In contrast, extracellular vesicles having a diameter of more than about 200 nm, but less than 400 nm, may be referred to as “medium extracellular vesicles,” and extracellular vesicles having a diameter of more than about 400 nm may be referred to as “large extracellular vesicles.” As used herein, the term “small extracellular vesicle fraction” (“sEV”) refers to a part, extract, or fraction, of secretome or conditioned medium, that is concentrated and/or enriched for small extracellular vesicles having a diameter of between about 50-200 nm. Such concentration and/or enrichment may be obtained using one or more of the purification, isolation, concentration, and/or enrichment, techniques disclosed herein.

The term “exosome” as used herein refers to an extracellular vesicle that is released from a cell upon fusion of the multivesicular body (MVB) (an intermediate endocytic compartment) with the plasma membrane.

“Exosome-like vesicles,” which have a common origin with exosomes, are typically described as having size and sedimentation properties that distinguish them from exosomes and, particularly, as lacking lipid raft microdomains. “Ectosomes,” as used herein, are typically neutrophil- or monocyte-derived microvesicles.

“Microparticles” as used herein are typically about 100-1000 nm in diameter and originate from the plasma membrane. “Extracellular membranous structures” also include linear or folded membrane fragments, e.g., from necrotic death, as well as membranous structures from other cellular sources, including secreted lysosomes and nanotubes.

As used herein, “apoptotic blebs or bodies” are typically about 1 to 5 μm in diameter and are released as blebs of cells undergoing apoptosis, i.e., diseased, unwanted and/or aberrant cells.

Within the class of extracellular vesicles, important components are “exosomes” themselves, which may be between about 40 to 50 nm and about 200 nm in diameter and being membranous vesicles, i.e., vesicles surrounded by a phospholipid bilayer, of endocytic origin, which result from exocytic fusion, or “exocytosis” of multivesicular bodies (MVBs). In some cases, exosomes can be between about 40 to 50 nm up to about 200 nm in diameter, such as being from 60 nm to 180 nm.

As used herein, the terms “secretome” and “secretome composition” interchangeably refer to one or more molecules and/or biological factors that are secreted by cells into the extracellular space (such as into a culture medium). A secretome or secretome composition may include, without limitation, extracellular vesicles (e.g., exosomes, microparticles, etc.), proteins, nucleic acids, cytokines, and/or other molecules secreted by cells into the extracellular space (such as into a culture medium). A secretome or secretome composition may be left unpurified or further processed (for example, components of a secretome or secretome composition may be present within culture medium, such as in a conditioned medium; or alternatively, components of a secretome or secretome composition may be purified, isolated, and/or enriched, from a culture medium or extract, part, or fraction thereof). A secretome or secretome composition may further comprise one or more substances that are not secreted from a cell (e.g., culture media, additives, nutrients, etc.). Alternatively, a secretome or secretome composition does not comprise one or more substances (or comprises only trace amounts thereof) that are not secreted from a cell (e.g., culture media, additives, nutrients, etc.).

As used herein, the term “conditioned medium” refers to a culture medium (or extract, part, or fraction thereof) in which one or more cells of interest have been cultured. Preferably, conditioned medium is separated from the cultured cells before use and/or further processing. The culturing of cells in culture medium may result in the secretion and/or accumulation of one or more molecules and/or biological factors (which may include, without limitation, extracellular vesicles (e.g., exosomes, microparticles, etc.), proteins, nucleic acids, cytokines, and/or other molecules secreted by cells into the extracellular space); the medium containing the one or more molecules and/or biological factors is a conditioned medium. Examples of methods of preparing conditioned media have been described in, for example, U.S. Pat. No. 6,372,494, which is incorporated by reference herein in its entirety.

As used herein, the term “cell culture” refers to cells grown under controlled condition(s) outside the natural environment of the cells. For instance, cells can be propagated completely outside of their natural environment (in vitro), or can be removed from their natural environment and the cultured (ex vivo). During cell culture, cells may survive in a non-replicative state, or may replicate and grow in number, depending on, for example, the specific culture media, the culture conditions, and the type of cells. An in vitro environment can be any medium known in the art that is suitable for maintaining cells in vitro, such as suitable liquid media or agar, for example.

The term “cell line” as used herein can refer to cultured cells that can be passaged at least one time without terminating.

The term “suspension” as used herein can refer to cell culture conditions in which cells are not attached to a solid support. Cells proliferating in suspension can be stirred while proliferating using an apparatus well known to those skilled in the art.

The term “monolayer” as used herein can refer to cells that are attached to a solid support while proliferating in suitable culture conditions. A small portion of cells proliferating in a monolayer under suitable growth conditions may be attached to cells in the monolayer but not to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel that has been supplemented with medium suitable for further cell proliferation.

As used herein, the terms “culture medium,” “growth medium” or “medium” are used interchangeably and refer to a composition that is intended to support the growth and survival of organisms. While culture media is often in liquid form, other physical forms may be used, such as, for example, a solid, semi-solid, gel, suspension, and the like.

As used herein, the term “serum-free,” in the context of a culture medium or growth medium, refers to a culture or growth medium in which serum is absent. Serum typically refers to the liquid component of clotted blood, after the clotting factors (e.g., fibrinogen and prothrombin) have been removed by clot formation. Serum, such as fetal bovine serum, is routinely used in the art as a component of cell culture media, as the various proteins and growth factors therein are particularly useful for the survival, growth, and division of cells.

As used herein, the term “basal medium” refers to an unsupplemented synthetic medium that may contain buffers, one or more carbon sources, amino acids, and salts. Depending on the application, basal medium may be supplemented with growth factors and supplements, including, but not limited to, additional buffering agents, amino acids, antibiotics, proteins, and growth factors useful, for instance, for promoting growth, or maintaining or changing differentiation status, of particular cell types (e.g., fibroblast growth factor-basic; bFGF).

As used herein, the terms “wild-type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

As used herein, the term “isolated” refers to material removed from its original environment, and is thus altered “by the hand of man” from its natural state.

As used herein, the term “enriched” means to selectively concentrate or increase the amount of one or more components in a composition, with respect to one or more other components. For instance, enrichment may include reducing or decreasing the amount of (e.g., removing or eliminating) unwanted materials; and/or may include specifically selecting or isolating desirable materials from a composition.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

As used herein, the terms “nucleic acid sequence,” “nucleotide sequence,” and “oligonucleotide” all refer to polymeric forms of nucleotides. As used herein, the term “polynucleotide” refers to a polymeric form of nucleotides that, when in linear form, has one 5′ end and one 3′ end, and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidates, methyl phosphonates, chiral-methyl phosphonates, 2-O-methyl ribonucleotides, peptide-nucleic acids (PNAs), Locked Nucleic Acid (LNA™) (Exiqon, Inc., Woburn, MA) nucleosides, glycol nucleic acid, bridged nucleic acids, and morpholino structures. Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA. Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide using algorithms having various weighting parameters. Sequence identity between two polynucleotides or two polypeptides can be determined using sequence alignment by various methods and computer programs (e.g., Exonerate, BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is often between about 90% identity and 100% identity over the length of the reference polynucleotide or polypeptide or query sequence, for example, about 90% identity or higher, about 91% identity or higher, about 92% identity or higher, about 93% identity or higher, about 94% identity or higher, about 95% identity or higher, about 96% identity or higher, about 97% identity or higher, about 98% identity or higher, or about 99% identity or higher, over the length of the reference polynucleotide or polypeptide or query sequence. Sequence identity can also be calculated for the overlapping region of two sequences where only a portion of the two sequences can be aligned.

A moderate degree of sequence identity between two polynucleotides or two polypeptides is often between about 80% identity to about 90% identity over the length of the reference polynucleotide or polypeptide or query sequence, for example, about 80% identity or higher, about 81% identity or higher, about 82% identity or higher, about 83% identity or higher, about 84% identity or higher, about 85% identity or higher, about 86% identity or higher, about 87% identity or higher, about 88% identity or higher, or about 89% identity or higher, but less than 90%, over the length of the reference polynucleotide or polypeptide or query sequence.

A low degree of sequence identity between two polynucleotides or two polypeptides is often between about 50% identity and 75% identity over the length of the reference polynucleotide or polypeptide or query sequence, for example, about 50% identity or higher, about 60% identity or higher, about 70% identity or higher, but less than 75% identity, over the length of the reference polynucleotide or polypeptide or query sequence.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Binding interactions can be characterized by a dissociation constant (Kd). “Binding affinity” refers to the strength of the binding interaction. An increased binding affinity is correlated with a lower Kd.

“Gene” as used herein refers to a polynucleotide sequence comprising exons and related regulatory sequences. A gene may further comprise introns and/or untranslated regions (UTRs).

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, a “different” or “altered” level of, for example, a characteristic or property, is a difference that is measurably different, and preferably, statistically significant (for example, not attributable to the standard error of the assay). In some embodiments, a difference, e.g., as compared to a control or reference sample, may be, for example, a greater than 10% difference, a greater than 20% difference, a greater than 30% difference, a greater than 40% difference, a greater than 50% difference, a greater than 60% difference, a greater than 70% difference, a greater than 80% difference, a greater than 90% difference, a greater than 2-fold difference; a greater than 5-fold difference; a greater than 10-fold difference; a greater than 20-fold difference; a greater than 50-fold difference; a greater than 75-fold difference; a greater than 100-fold difference; a greater than 250-fold difference; a greater than 500-fold difference; a greater than 750-fold difference; or a greater than 1,000-fold difference, for example.

As used herein, the term “between” is inclusive of end values in a given range (e.g., between about 1 and about 50 nucleotides in length includes 1 nucleotide and 50 nucleotides).

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology.

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description.

The terms “effective amount” or “therapeutically effective amount” of a composition or agent, such as a therapeutic composition as provided herein, refers to a sufficient amount of the composition or agent to provide the desired response. Such responses will depend on the particular disease in question.

“Transformation” as used herein refers to the insertion of an exogenous polynucleotide into a host cell, irrespective of the method used for insertion. For example, transformation can be by direct uptake, transfection, infection, and the like. The exogenous polynucleotide may be maintained as a nonintegrated vector, for example, an episome, or, alternatively, may be integrated into the host genome.

### Obtaining Progenitor Cells

The present disclosure relates, in part, to methods and assays for analyzing an activity, functionality, and/or potency, of conditioned media; or of a secretome-, extracellular vesicle-, and/or a small extracellular vesicle-enriched fraction (sEV)-containing composition. In some embodiments, the conditioned media, or the secretome-, extracellular vesicle-, and/or a small extracellular vesicle-enriched fraction (sEV)-containing composition, may be obtained from one or more cells in culture, such as cultured progenitor cells. However, other types of cells may also be used, including, for example, end-stage differentiated cells, pluripotent stem cells, etc.

Progenitor cells suitable for producing a conditioned media, or a secretome-, extracellular vesicle-, and/or a small extracellular vesicle-enriched fraction (sEV)-containing composition, may be, for example, isolated from a subject or tissue; or generated from pluripotent stem cells, such as from embryonic stem (ES) cells or induced pluripotent stem cells (iPSCs). In some embodiments, an activity, functionality, and/or potency, can be assessed for a conditioned media, or a secretome-, extracellular vesicle-, and/or a small extracellular vesicle-enriched fraction (sEV)-containing composition, produced from progenitor cells that have been differentiated from iPSC cells.

### Generation of iPSC Cells

iPSC cells may be obtained from, for example, somatic cells, including human somatic cells. The somatic cell may be derived from a human or non-human animal, including, for example, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like.

In some embodiments, the somatic cell is selected from keratinizing epithelial cells, mucosal epithelial cells, exocrine gland epithelial cells, endocrine cells, liver cells, epithelial cells, endothelial cells, fibroblasts, muscle cells, cells of the blood and the immune system, cells of the nervous system including nerve cells and glial cells, pigment cells, and progenitor cells, including hematopoietic stem cells. The somatic cell may be fully differentiated (specialized), or may be less than fully differentiated. For instance, undifferentiated progenitor cells that are not PSCs, including somatic stem cells, and finally differentiated mature cells, can be used. The somatic cell may be from an animal of any age, including adult and fetal cells.

The somatic cell may be of mammalian origin. Allogeneic or autologous stem cells can be used, if for example, the secretome (or extracellular vesicles) from a progenitor cell thereof is used for administration in vivo. In some embodiments, iPSCs are not MHC-/HLA-matched to a subject. In some embodiments, iPSCs are MHC-/HLA-matched to a subject. In embodiments, for example, where iPSCs are to be used to produce PSC-derived progenitor cells (to obtain a secretome, or extracellular vesicles, for therapeutic use in a subject), somatic cells may be obtained from the subject to be treated, or from another subject with the same or substantially the same HLA type as that of the subject. Somatic cells can be cultured before nuclear reprogramming, or can be reprogrammed without culturing after isolation, for example.

To introduce reprogramming factors into somatic cells, for example, viral vectors may be used, including, e.g., vectors from viruses such as SV40, adenovirus, vaccinia virus, adeno-associated virus, herpes viruses including HSV and EBV, Sindbis viruses, alphaviruses, human herpesvirus vectors (HHV) such as HHV-6 and HHV-7, and retroviruses. Lentiviruses include, but are not limited to, Human Immunodeficiency Virus type 1 (HIV-1), Human Immunodeficiency Virus type 2 (HIV-2), Simian Immunodeficiency Virus (SIV), Feline Immunodeficiency Virus (FIV), Equine Infectious Anaemia Virus (EIAV), Bovine Immunodeficiency Virus (BIV), Visna Virus of sheep (VISNA) and Caprine Arthritis-Encephalitis Virus (CAEV). Lentiviral vectors are capable of infecting non-dividing cells and can be used for both in vivo and in vitro gene transfer and expression of nucleic acid sequences. A viral vector can be targeted to a specific cell type by linkage of a viral protein, such as an envelope protein, to a binding agent, such as an antibody, or a particular ligand (for targeting to, for instance, a receptor or protein on or within a particular cell type).

In some embodiments, a viral vector, such as a lentiviral vector, can integrate into the genome of the host cell. The genetic material thus transferred is then transcribed and possibly translated into proteins inside the host cell. In other embodiments, viral vectors are used that do not integrate into the genome of a host cell.

A viral gene delivery system can be an RNA-based or DNA-based viral vector. An episomal gene delivery system can be a plasmid, an Epstein-Barr virus (EBV)-based episomal vector, a yeast-based vector, an adenovirus-based vector, a simian virus 40 (SV40)-based episomal vector, a bovine papilloma virus (BPV)-based vector, or a lentiviral vector, for example.

Somatic cells can be reprogrammed to produce induced pluripotent stem cells (iPSCs) using methods known to one of skill in the art. One of skill in the art can readily produce induced pluripotent stem cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; and 8,268,620, all of which are incorporated herein by reference.

Generally, reprogramming factors which can be used to create induced pluripotent stem cells, either singly, in combination, or as fusions with transactivation domains, include, but are not limited to, one or more of the following genes: Oct4 (Oct3/4, Pou5f1), Sox (e.g., Sox1, Sox2, Sox3, Sox18, or Sox15), Klf (e.g., Klf4, Klf1, Klf3, Klf2 or Klf5), Myc (e.g., c-myc, N-myc or L-myc), nanog, or LIN28. As examples of sequences for these genes and proteins, the following accession numbers are provided: Mouse MyoD: M84918, NM_010866; Mouse Oct4 (POU5F1): NM_013633; Mouse Sox2: NM_011443; Mouse Klf4: NM_010637; Mouse c-Myc: NM_001177352, NM_001177353, NM_001177354 Mouse Nanog: NM_028016; Mouse Lin28: NM_145833: Human MyoD: NM_002478; Human Oct4 (POU5F1): NM_002701, NM_203289, NM_001173531; Human Sox2: NM_003106; Human Klf4: NM_004235; Human c-Myc: NM_002467; Human Nanog: NM_024865; and/or Human Lin28: NM_024674. Also contemplated are sequences similar thereto, including those having at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% sequence identity. In some embodiments, at least three, or at least four, of Klf4, c-Myc, Oct3/4, Sox2, Nanog, and Lin28 are utilized. In other embodiments, Oct3/4, Sox2, c-Myc and Klf4 is utilized.

Exemplary reprogramming factors for the production of iPSCs include (1) Oct3/4, Klf4, Sox2, L-Myc (Sox2 can be replaced with Sox1, Sox3, Sox15, Sox17 or Sox18; Klf4 is replaceable with Klf1, Klf2 or Klf5); (2) Oct3/4, Klf4, Sox2, L-Myc, TERT, SV40 Large T antigen (SV41LT); (3) Oct3/4, Klf4, Sox2, L-Myc, TERT, human papilloma virus (HPV)16 E6; (4) Oct3/4, Klf4, Sox2, L-Myc, TERT, HPV16 E7 (5) Oct3/4, Klf4, Sox2, L-Myc, TERT, HPV16 E6, HPV16 E7; (6) Oct3/4, Klf4, Sox2, L-Myc, TERT, Bmi1; (7) Oct3/4, Klf4, Sox2, L-Myc, Lin28; (8) Oct3/4, Klf4, Sox2, L-Myc, Lin28, SV40LT; (9) Oct3/4, Klf4, Sox2, L-Myc, Lin28, TERT, SV40LT; (10) Oct3/4, Klf4, Sox2, L-Myc, SV40LT; (11) Oct3/4, Esrrb, Sox2, L-Myc (Esrrb is replaceable with Esrrg); (12) Oct3/4, Klf4, Sox2; (13) Oct3/4, Klf4, Sox2, TERT, SV41LT; (14) Oct3/4, Klf4, Sox2, TERT, HPV16 E6; (15) Oct3/4, Klf4, Sox2, TERT, HPV16 E7; (16) Oct3/4, Klf4, Sox2, TERT, HPV16 E6, HPV16 E7; (17) Oct3/4, Klf4, Sox2, TERT, Bmi1; (18) Oct3/4, Klf4, Sox2, Lin28 (19) Oct3/4, Klf4, Sox2, Lin28, SV41LT; (20) Oct3/4, Klf4, Sox2, Lin28, TERT, SV40LT; (21) Oct3/4, Klf4, Sox2, SV40LT; or (22) Oct3/4, Esrrb, Sox2 (Esrrb is replaceable with Esrrg).

iPSCs typically display the characteristic morphology of human embryonic stem cells (hESCs), and express the pluripotency factor, NANOG. Embryonic stem cell specific surface antigens (SSEA-3, SSEA-4, TRA1-60, TRA1-81) may also be used to identify fully reprogrammed human cells. Additionally, at a functional level, PSCs, such as ESCs and iPSCs, also demonstrate the ability to differentiate into lineages from all three embryonic germ layers, and form teratomas in vivo (e.g., in SCID mice).

### Differentiating PSCs to Generate Progenitor Cells

The present disclosure further contemplates differentiating PSCs, including ESCs and iPSCs, into progenitor cells. Such progenitor cells can then be used to produce a secretome (and extracellular vesicles) of the present disclosure.

Progenitor cells of the present disclosure include, for example, hematopoietic progenitor cells, myeloid progenitor cells, neural progenitor cells; pancreatic progenitor cells, cardiac progenitor cells, cardiomyocyte progenitor cells, cardiovascular progenitor cells, renal progenitor cells, skeletal myoblasts, satellite cells, intermediate progenitor cells formed in the subventricular zone, radial glial cells, bone marrow stromal cells, periosteum cells, endothelial progenitor cells, blast cells, boundary caop cells, and mesenchymal stem cells. Methods for differentiating pluripotent stem cells to progenitor cells, and for culturing and maintaining progenitor cells, are known in the art, such as those described in U.S. Provisional Patent Application No. 63/243,606 entitled “Methods for the Production of Committed Cardiac Progenitor Cells,” which is incorporated by reference herein in its entirety.

Obtained progenitor cells can then be cultured, and the resulting culture medium can then be recovered (and in some instances further processed), to provide a conditioned media, or a secretome-, extracellular vesicle-, and/or a small extracellular vesicle-enriched fraction (sEV)-containing composition. The present disclosure also contemplates engineered extracellular vesicles. For example, a conditioned media, or a secretome-, extracellular vesicle-, and/or a small extracellular vesicle-enriched fraction (sEV)-containing composition, may be recovered from engineered cells. Additionally, or alternatively, the extracellular vesicles themselves may be engineered, before, during, and/or after, their recovery.

For instance, recovered culture medium can be concentrated, purified, refrigerated, frozen, cryopreserved, lyophilized, sterilized, etc. In some embodiments, the recovered, conditioned medium may be pre-cleared to remove particulates of greater than a certain size. For instance, the recovered, conditioned medium may be pre-cleared by one or more centrifugation and/or filtration techniques.

In some embodiments, the recovered, conditioned medium is further processed to obtain a particular extract or fraction of the recovered, conditioned medium. For instance, the recovered, conditioned medium may be further processed to separate a small extracellular vesicle-enriched fraction (sEV) therefrom. An sEV fraction may be separated from the recovered, conditioned medium (or from a previously processed extract or fraction thereof) by one or more techniques such as centrifugation, ultracentrifugation, filtration, ultrafiltration, gravity, sonication, density-gradient ultracentrifugation, tangential flow filtration, size-exclusion chromatography, ion-exchange chromatography, affinity capture, polymer-based precipitation, or organic solvent precipitation, for example.

Any of the above-described processing techniques can be performed on recovered, conditioned medium (or a previously processed extract or fraction thereof) that is fresh, or has previously been frozen and/or refrigerated, for example.

In some embodiments, the sEV fraction to be analyzed by the methods and assays herein is CD63⁺, CD81⁺, and/or CD9⁺. The sEV fraction may contain one or more extracellular vesicle types, such as, for example, one or more of exosomes, microparticles, and extracellular vesicles. The sEV fraction may also contain secreted proteins (enveloped and/or unenveloped). Extracellular vesicles within conditioned media or sEV fractions of the present disclosure may contain, for example, one or more components selected from tetraspanins (e.g., CD9, CD63 and CD81), ceramide, MHC class I, MHC class II, integrins, adhesion molecules, phosphatidylserine, sphingomyelin, cholesterol, cytoskeletal proteins (e.g., actin, gelsolin, myosin, tubulin), enzymes (e.g., catalase, GAPDH, nitric oxide synthase, LT synthases), nucleic acids (e.g., RNA, miRNA), heat shock proteins (e.g., HSP70 and HSP90), exosome biogenesis proteins (ALIX, Tsg101), LT, prostaglandins, and S100 proteins.

In some embodiments, the presence of desired extracellular vesicle types in a fraction can be determined, for example, by electromicroscopy; by nanoparticle tracking analysis (to determine the sizes of particles in the fraction); and/or by confirming the presence of one or more markers associated with a desired extracellular vesicle type(s). For instance, a fraction of recovered, conditioned media can be analyzed for the presence of a desired extracellular vesicle type(s) by detecting the presence of one or more markers in the fraction, such as, for example, CD9, CD63 and/or CD81.

In some embodiments, an sEV formulation or composition is positive for CD9, CD63 and CD81 (canonical EV markers), and is positive for the cardiac-related markers CD49e, ROR1, SSEA-4, MSCP, CD146, CD41b, CD24, CD44, CD236, CD133/1, CD29 and CD142. In some embodiments, an sEV formulation or composition contains a lesser amount of one or more markers selected from the group consisting of CD3, CD4, CD8, HLA-DRDPDQ, CD56, CD105, CD2, CD1c, CD25, CD40, CD11c, CD86, CD31, CD20, CD19, CD209, HLA-ABC, CD62P, CD42a and CD69, as compared to the amount of CD9, CD63 and/or CD81 in the sEV formulation or composition. In some embodiments, an sEV formulation or composition contains an undetectable amount of (e.g., by MACSPlex assay, by immunoassay, etc.), or is negative for, one or more markers selected from the group consisting of CD19, CD209, HLA-ABC, CD62P, CD42a and CD69.

In some embodiments, the sEV formulation or composition is at least one of the following: an sEV formulation or composition that has been enriched for extracellular vesicles having a diameter of between about 50-200 nm or between 50-200 nm; an sEV formulation or composition that has been enriched for extracellular vesicles having a diameter of between about 50-150 nm or between 50-150 nm; an sEV formulation or composition that is substantially free or free of whole cells; and an sEV formulation or composition that is substantially free of one or more culture medium components (e.g., phenol-red).

### Assays for Determining Secretome and Extracellular Vesicle Activity, Functionality, and/or Potency

The present disclosure encompasses methods for analyzing an activity, functionality, characteristic, and/or potency, of conditioned media; or of a secretome-, extracellular vesicle-, and/or sEV-containing composition. The activity, functionality, characteristic, and/or potency, of conditioned media; or of a secretome-, extracellular vesicle-, and/or sEV-containing composition, can be assessed by various techniques, depending on, for example, the type of cell(s) used to produce the conditioned media or composition; and the desired use of the conditioned media or composition.

For instance, an activity, functionality, characteristic, and/or potency, of conditioned media; or of a secretome-, extracellular vesicle-, and/or sEV-containing composition, can be assessed by administering the conditioned media, secretome-, extracellular vesicle-, and/or sEV-containing composition, to target cells in vitro, ex vivo, or in vivo. One or more properties of the target cells can then be analyzed, such as, for example, cell viability, hypertrophy, cell health, cell adhesion, cell physiology, ATP content, cell number, cell growth. and cell morphology (to determine the activity, functionality, characteristic, and/or potency, of the conditioned media; or of the secretome-, extracellular vesicle-, and/or sEV-containing composition).

In methods and assays of the present disclosure, an activity, functionality, characteristic, and/or potency, of conditioned media; or of a secretome-, extracellular vesicle-, and/or sEV-containing composition, can be assessed by a method comprising administering the conditioned media, or the secretome-, extracellular vesicle-, and/or sEV-containing composition, to target cells cultured under at least one stress-inducing condition, and analyzing at least one property of the cells. The one or more properties of the target cells that may be analyzed can be selected from, for instance, cell migration, cell survival, cell viability, hypertrophy, cell health, cell adhesion, cell physiology, ATP content, cell number, cell growth, and cell morphology. In some embodiments, at least 2, at least 3, at least 4, at least 5, at least 6, at least 7, at least 8, at least 9, or at least 10, properties of the target cells are measured.

In a first method thereof, target cells are cultured in a pre-treatment medium under at least one stress-inducing condition, followed by administering a conditioned medium or a secretome-, extracellular vesicle-, and/or sEV-containing composition, to the cell culture. The target cells are then cultured in the presence of the conditioned medium or the secretome-, extracellular vesicle-, and/or sEV-containing composition, and at least one property of the cultured cells is measured one or more times during the culturing. In some embodiments, the at least one property is measured multiple times during the culturing in the presence of the conditioned medium or the secretome-, extracellular vesicle-, and/or sEV-containing composition (such as, for example, 5 minutes to 10 hours apart from each other; 10 minutes to 4 hours apart from each other; or 30 minutes to 2 hours apart from each other). In some embodiments, the at least one property is measured at least 2, at least 3, at least 4, at least 5, at least 6, at least 7, at least 8, at least 9, at least 10, at least 20, at least 30, at least 50, at least 100, or at least 500, times during the culturing in the presence of the conditioned medium or the secretome-, extracellular vesicle-, and/or sEV-containing composition.

In some embodiments of this first method, the culturing in the presence of the conditioned medium or the secretome-, extracellular vesicle-, and/or sEV-containing composition occurs in the presence of the at least one stress-inducing condition. In other embodiments of this first method, the culturing in the presence of the conditioned medium or the secretome-, extracellular vesicle-, and/or sEV-containing composition occurs in the absence of the at least one stress-inducing condition.

In some embodiments of this first method, the pre-treatment medium is removed from the cells before the culturing in the presence of the conditioned medium or the secretome-, extracellular vesicle-, and/or sEV-containing composition. Thus, in embodiments of the first method where the at least one stress-inducing condition is provided by the pre-treatment medium (e.g., by a stress-inducing agent present in the pre-treatment medium), the culturing in the presence of the conditioned medium or the secretome-, extracellular vesicle-, and/or sEV-containing composition occurs in the absence of the at least one stress-inducing condition.

In other embodiments of this first method, the pre-treatment medium is not removed from the cells before the culturing in the presence of the conditioned medium or the secretome-, extracellular vesicle-, and/or sEV-containing composition. Thus, in embodiments of the first method where the at least one stress-inducing condition is provided by the pre-treatment medium (e.g., by a stress-inducing agent present in the pre-treatment medium), the culturing in the presence of the conditioned medium or the secretome-, extracellular vesicle-, and/or sEV-containing composition occurs in the presence of the at least one stress-inducing condition.

In a second method, target cells are cultured in a pre-treatment medium, followed by administering a conditioned medium or a secretome-, extracellular vesicle-, and/or sEV-containing composition (and optionally thereafter, culturing the target cells in the presence of the conditioned medium or the secretome-, extracellular vesicle-, and/or sEV-containing composition). Following this, the target cells are then cultured under at least one stress-inducing condition, and at least one property of the cultured cells is measured one or more times during the culturing under the at least one stress-inducing condition (which, in some embodiments, may occur in the presence of the conditioned medium or the secretome-, extracellular vesicle-, and/or sEV-containing composition). In some embodiments, the at least one property is measured multiple times during the culturing under the at least one stress-inducing condition, such as, for example, 5 minutes to 10 hours apart from each other; 10 minutes to 4 hours apart from each other; or 30 minutes to 2 hours apart from each other. In some embodiments, the at least one property is measured at least 2, at least 3, at least 4, at least 5, at least 6, at least 7, at least 8, at least 9, at least 10, at least 20, at least 30, at least 50, at least 100, or at least 500, times during the culturing under the at least one stress-inducing condition.

In some embodiments of this second method, the target cells are cultured in the presence of the conditioned medium or the secretome-, extracellular vesicle-, and/or sEV-containing composition, before being cultured under the at least one stress-inducing condition. In other embodiments of this second method, the target cells are not cultured in the presence of the conditioned medium or the secretome-, extracellular vesicle-, and/or sEV-containing composition, before being cultured under the at least one stress-inducing condition. In some embodiments of this second method, the conditioned medium or the secretome-, extracellular vesicle-, and/or sEV-containing composition is removed from the target cells before the target cells are cultured in the presence of the at least one stress-inducing condition.

In some embodiments of the above first and second methods, the stress-inducing condition is culturing in the presence of a cellular stress agent. In some embodiments of the second method, the cellular stress agent is co-administered to the target cells with the conditioned medium or the secretome-, extracellular vesicle-, and/or sEV-containing composition.

In some embodiments of the above first and second methods, the cellular stress agent is one or more chemotherapeutic agents, and/or one or more apoptosis-inducing agents.

Chemotherapeutic agents may be selected from, for example, antimetabolites, alkylating agents, topoisomerase inhibitors, mitotic inhibitors, antitumor antibiotics, kinase inhibitors (including protein kinase inhibitors), anthracyclines, platinums, plant alkaloids, nitrosureas, cytoskeletal disruptors, epothilones, histone deacetylase inhibitors, nucleotide analogs and precursor analogs, peptide antibiotics, and retinoids.

The one or more apoptosis-inducing agents may be selected from, for example, doxorubicin, staurosporine, etoposide, camptothecin, paclitaxel, vinblastine, gambogic acid, daunorubicin, tyrphostins, thapsigargin, okadaic acid, mifepristone, colchicine, ionomycin, 24(S)-hydroxycholesterol, cytochalasin D, brefeldin A, raptinal, carboplatin, C2 ceramide, actinomycin D, rosiglitazone, kaempferol, berberine chloride, bioymifi, betulinic acid, tamoxifen, embelin, phytosphingosine, mitomycin C, birinapant, anisomycin, genistein, cycloheximide, and the like, and derivatives and combinations thereof.

In some embodiments, the apoptosis-inducing agent is an indolocarbazole. In some embodiments, the apoptosis-inducing agent is an indolo(2,3-a)pyrrole(3,4-c)carbazole. In some embodiments, the apoptosis-inducing agent is staurosporine, or a derivative thereof. In other embodiments, the apoptosis-inducing agent is doxorubicin, or a derivative thereof.

In some embodiments of the first and second methods, the at least one property measured is viability of the cultured cells. The viability may be measured, for example, using a DNA-labeling dye or a nuclear-staining dye. In some embodiments thereof, the DNA-labeling dye or the nuclear-staining dye is a fluorescent dye, such as a far-red fluorescent dye.

In some embodiments of the first and second methods, the at least one property measured is cell adhesion, cell number, cell growth, and/or cell morphology, and wherein the cell adhesion, cell number, cell growth, and/or cell morphology, is determined by measuring electrical impedance across a culture vessel surface in the culture.

In embodiments of the first and second methods, the target cells can be cultured in the pre-treatment medium for differing lengths of time. For instance, the target cells can be cultured in the pre-treatment medium for 30 minutes to 10 hours, 1 hour to 5 hours, or more than, less than, or about, 1 hour, 2 hours, 3 hours, 4 hours, or 5 hours.

In embodiments of the first and second methods, the target cells are cultured with the conditioned medium or the secretome-, extracellular vesicle-, and/or sEV-containing composition, for at least 30 minutes, at least 1 hour, at least 2 hours, at least 4 hours, at least 6 hours, at least 8 hours, at least 12 hours, at least 18 hours, at least 24 hours, at least 36 hours, or at least 48 hours.

In some embodiments of the first and second methods, the target cells are cultured in vitro prior to culturing in the pre-treatment medium. For instance, the target cells may be cultured in vitro for between 1-21 days, between 3-17 days, between 5-14 days, or less than 20 days, less than 18 days, less than 16 days, less than 14 days, less than 12 days, less than 10 days, less than 8 days, less than 6 days, less than 4 days, or less than 2 days, prior to culturing in the pre-treatment medium. In certain embodiments in which the target cells are cultured in vitro prior to culturing in the pre-treatment medium, the target cells are supplied with fresh culture medium prior to culturing in the pre-treatment medium. For instance, the target cells may be supplied with fresh culture medium 6-72 hours, 8-60 hours, 10-48 hours, or 12-36 hours, prior to culturing in the pre-treatment medium.

In embodiments of the first and second methods, the culturing of the target cells may be two-dimensional or three-dimensional cell culturing. For instance, in some embodiments, the culture vessel used for culturing may be a flask, flask for tissue culture, hyperflask, dish, petri dish, dish for tissue culture, multi dish, micro plate, micro-well plate, multi plate, multi-well plate, micro slide, chamber slide, tube, tray, CellSTACK® Chambers, culture bag, roller bottle, bioreactor, stirred culture vessel, spinner flask, or a vertical wheel bioreactor, for example.

In embodiments in which culturing comprises two-dimensional cell culture, such as on the surface of a culture vessel, the culture surface (to which the cells are intended to adhere) may be coated with one or more substances that promote cell adhesion. Such substances useful for enhancing attachment to a solid support include, for example, type I, type II, and type IV collagen, concanavalin A, chondroitin sulfate, fibronectin, fibronectin-like polymers, gelatin, laminin, poly-D and poly-L-lysine, Matrigel, thrombospondin, and/or vitronectin.

In embodiments of the first and second methods, the at least one property may also be analyzed with reference to one or more control samples.

For instance, the first and second methods may further comprise culturing positive control cells (e.g., in parallel), wherein the positive control cells are not administered the conditioned medium or the secretome-, extracellular vesicle-, and/or sEV-containing composition, and are not cultured under the at least one stress-inducing condition. Thus, in embodiments in which the stress inducing condition is the presence of an apoptosis-inducing agent, the positive control cells are not administered the apoptosis-inducing agent.

The first and second methods may comprise culturing negative control cells (e.g., in parallel), wherein the negative control cells are not administered the conditioned medium or the secretome-, extracellular vesicle-, and/or sEV-containing composition. In some embodiments, the negative control cells comprise negative control cells subjected to the same steps as the target cells, except that they are not administered the conditioned medium or the secretome-, extracellular vesicle-, and/or sEV-containing composition.

In certain embodiments, the negative control cells comprise negative control cells cultured in the pre-treatment medium under the at least one stress-inducing condition. The at least one property measured in the target cells may also then be measured in the negative control cells, during and/or after they are cultured in the pre-treatment medium under the at least one stress-inducing condition.

In some embodiments, the negative control cells comprise negative control cells to which a mock conditioned medium or a mock secretome-, extracellular vesicle-, and/or sEV-containing composition is added. In specific embodiments thereof, the mock conditioned medium or the mock secretome-, extracellular vesicle-, and/or sEV-containing composition is produced by omitting cells from the process of producing a conditioned medium or a secretome-, extracellular vesicle-, and/or sEV-containing composition.

The use of such a negative control(s) allows an activity, functionality and/or potency, of a conditioned medium or a secretome-, extracellular vesicle-, and/or sEV-containing composition to be evaluated.

For instance, where the at least one property measured is viability of the cultured cells, a conditioned medium or a secretome-, extracellular vesicle-, and/or sEV-containing composition may be determined to have an activity, functionality, potency (and/or exhibit a therapeutic effect), when the viability of the target cells is higher than the viability of negative control cells which have also been subjected to the at least one stress-inducing condition.

Alternatively, for instance, where the at least one property measured is cell adhesion, cell growth, and/or cell number, and wherein the cell adhesion, cell growth, and/or cell number is determined by measuring electrical impedance across a culture vessel surface in the culture, a conditioned medium or a secretome-, extracellular vesicle-, and/or sEV-containing composition may be determined to have an activity, functionality, potency (and/or exhibit a therapeutic effect), when the electrical impedance across a culture vessel surface in the culture is higher than the electrical impedance across a culture vessel surface in a culture of negative control cells which have also been subjected to the at least one stress-inducing condition.

Any one or more samples, and/or any one or more positive and/or negative controls, may be performed in replicate, such as, for example, in duplicate, in triplicate, etc. In some embodiments thereof in which cell viability is measured, and where replicate cultures are performed, the number of positive control cells in the replicate cultures may be averaged to produce an average maximum cell number (and the number of target cells in each replicate test culture may be normalized to the average maximum cell number, to calculate cell viability).

In some embodiments, assays known in the art may be used in conjunction with the methods and assays of the present disclosure, to further determine, validate, and/or confirm the activity, functionality, and/or potency, of conditioned media; or of a secretome-, extracellular vesicle-, and/or sEV-containing composition.

For instance, for conditioned media; or for a secretome-, extracellular vesicle-, and/or sEV-containing composition, obtained from cardiomyocyte progenitor cells, the activity, functionality, and/or potency thereof may be further measured using a known cardiomyocyte viability assay, such as described in El Harane et al. (Eur. Heart J., 2018, 39(20): 1835-1847).

Specifically, serum-deprived cardiac myoblasts (e.g., H9c2 cells) may be contacted with conditioned media; or a secretome-, extracellular vesicle-, and/or sEV-containing composition, and the viability of the cells measured thereafter. In some embodiments of this assay, the cells are deprived of serum before administering the conditioned media or the secretome-, extracellular vesicle-, and/or sEV-containing composition. In other embodiments of this assay, the cells are deprived of serum after administering the conditioned media or the secretome-, extracellular vesicle-, and/or sEV-containing composition. In some embodiments of this assay, the cells are deprived of serum before and after administering the conditioned media or the secretome-, extracellular vesicle-, and/or sEV-containing composition.

Another assay known in the art which may be used in conjunction with the methods and assays of the present disclosure is a HUVEC scratch wound healing assay. In HUVEC scratch wound healing assays, HUVEC cells are cultured on a culture surface, and the cultured cell layer(s) is then scratched; angiogenic activity of a conditioned media or a secretome-, extracellular vesicle-, and/or sEV-containing composition can then be determined by the capacity of the conditioned media or the secretome-, extracellular vesicle-, and/or sEV-containing composition to produce closure of the wound under serum-free conditions.

To more accurately compare an activity, functionality, characteristic, and/or potency, between different conditioned media or secretome-, extracellular vesicle-, and/or sEV-containing compositions, it may be beneficial to determine the amount of the conditioned medium or the secretome-, extracellular vesicle-, and/or sEV-containing composition, added (or to be added) to target cells. This can be determined, for example, based on one or more of: the amount of secreting cells that produced the secretome; the protein content of the secretome; the RNA content of the secretome; the exosome amount of the secretome; and the number of particles in the secretome.

### Therapeutic Compositions and Applications

Conditioned media, or a secretome-, extracellular vesicle-, and/or a small extracellular vesicle-enriched fraction (sEV)-containing composition, can be analyzed by the methods and assays of the present disclosure to identify whether the media or composition contains the desired activity, functionality, and/or potency. If a media or composition exhibits the desired activity, functionality, and/or potency using a method and/or assay of the present disclosure, such a media or composition may be formulated or adapted for therapeutic use.

For instance, the tested media or composition may be a sample from a larger batch of conditioned media, or from a larger batch of a secretome-, extracellular vesicle-, and/or a small extracellular vesicle-enriched fraction (sEV)-containing composition. In such a case, all or a portion of the remaining media or composition from the batch may be formulated or adapted for therapeutic use.

Alternatively, the tested media or composition may be representative of other batches of conditioned media, or other batches of a secretome-, extracellular vesicle-, and/or a small extracellular vesicle-enriched fraction (sEV)-containing composition (because, for example, they were produced in parallel under similar, or the same, conditions). In such instances, the analysis results for the tested media or composition may serve as an indicator that the other batches of conditioned media, or the other batches of secretome-, extracellular vesicle-, and/or a small extracellular vesicle-enriched fraction (sEV)-containing composition, are suitable to be formulated or adapted for therapeutic use.

The methods and assays of the present disclosure may also be used to determine whether a particular process(es) for producing conditioned media, or for producing a secretome-, extracellular vesicle-, and/or a small extracellular vesicle-enriched fraction (sEV)-containing composition, yields a media or composition with the desired activity, functionality, or potency.

Accordingly, based on the analysis results of the methods and assays of the present disclosure, secretome-, extracellular vesicle-, and sEV-containing compositions, may be identified, selected, and/or formulated, for use as therapeutic agents (e.g., for administering an effective amount thereof to a subject in need thereof).

The identified, selected, and/or formulated secretome-, extracellular vesicle-, and/or sEV-containing compositions may be used to treat various tissues, including, without limitation, cardiac tissue, brain or other neural tissue, skeletal muscle tissue, pulmonary tissue, arterial tissue, capillary tissue, renal tissue, hepatic tissue, tissue of the gastrointestinal tract, epithelial tissue, connective tissue, tissue of the urinary tract, etc. The tissue to be treated may be damaged or fully or partly non-functional due to an injury, age-related degeneration, acute or chronic disease, cancer, or infection, for example. Such tissues may be treated, for example, by intravenous administration of a secretome-, extracellular vesicle-, and/or sEV-containing composition. Alternatively, the identified, selected, and/or formulated secretome-, extracellular vesicle-, and/or sEV-containing compositions may be used to pre-treat a patient prior to predicted or expected tissue injury. For example, the identified, selected, and/or formulated secretome-, extracellular vesicle-, and/or sEV-containing compositions may be administered prior to chemotherapy, to help prevent, for example, heart damage.

The identified, selected, and/or formulated secretome-, extracellular vesicle-, and/or sEV-containing compositions may be used to treat diseases such as myocardial infarction, stroke, heart failure, and critical limb ischemia, for example. In some embodiments, secretome-, extracellular vesicle-, and/or sEV-containing compositions may be used to treat heart failure which has one or more of the following characteristics: is acute, chronic, ischemic, non-ischemic, with ventricular dilation, or without ventricular dilation. In some embodiments, compositions of the present disclosure may be used to treat heart failure selected from the group consisting of ischemic heart disease, cardiomyopathy, myocarditis, hypertrophic cardiomyopathy, diastolic hypertrophic cardiomyopathy, dilated cardiomyopathy, and post-chemotherapy induced heart failure. In some embodiments, compositions of the present disclosure may be used to treat diseases such as congestive heart failure, heart disease, ischemic heart disease, valvular heart disease, connective tissue diseases, viral or bacterial infection, cardiomyopathy, myopathy, myocarditis, hypertrophic cardiomyopathy, diastolic hypertrophic cardiomyopathy, dystrophinopathy, liver disease, renal disease, sickle cell disease, diabetes, and neurological diseases. It will be recognized that a suitable progenitor cell type(s) may be selected depending on the disease to be treated, or the tissue to be targeted.

For example, in some embodiments, the identified, selected, and/or formulated secretome-, extracellular vesicle-, and/or sEV-containing composition may be used to treat a subject with a cardiac disease, such as acute myocardial infarction or heart failure. The identified, selected, and/or formulated secretome-, extracellular vesicle-, and/or sEV-containing composition may be produced, for example, from cardiomyocyte progenitor cells, cardiac progenitor cells, mesenchymal stem cells, and/or cardiovascular progenitor cells.

The identified, selected, and/or formulated secretome-, extracellular vesicle-, and/or sEV-containing composition may also be used to improve the functioning or performance of a tissue. For instance, an improvement in angiogenesis, or an improvement in cardiac performance, may be effected by delivering an identified, selected, and/or formulated secretome-, extracellular vesicle-, and/or sEV-containing composition produced from cardiomyocyte progenitor cells, cardiac progenitor cells, and/or cardiovascular progenitor cells, to a subject in need thereof.

The administration may comprise administration at a tissue or organ site that is the same as the target tissue. The administration may, additionally or alternatively, comprise administration at a tissue or organ site that is different from the target tissue. Such administration may include, for example, intravenous administration.

The identified, selected, and/or formulated secretome-, extracellular vesicle-, and/or sEV-containing composition may be administered with a pharmaceutically-acceptable diluent, carrier, or excipient. Such a composition may also contain, in some embodiments, pharmaceutically acceptable concentrations of one or more of a salt, buffering agent, preservative, or other therapeutic agent. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other nontoxic compatible substances employed in pharmaceutical formulations. For instance, in some embodiments, a secretome-, extracellular vesicle-, and/or sEV-containing composition, may be formulated with a biomaterial, such as an injectable biomaterial. Exemplary injectable biomaterials are described, for example, in WO 2018/046870, incorporated by reference herein in its entirety.

The identified, selected, and/or formulated secretome-, extracellular vesicle-, and/or sEV-containing composition may be administered in an effective amount, such as a therapeutically effective amount, depending on the purpose. An effective amount will depend upon a variety of factors, including the material selected for administration, whether the administration is in single or multiple doses, and individual patient parameters including age, physical condition, size, weight, and the stage of disease. These factors are well known to those of ordinary skill in the art.

Any appropriate route of administration may be employed, for example, administration may be parenteral, intravenous, intra-arterial, subcutaneous, intratumoral, intramuscular, intracranial, intraorbital, ophthalmic, intraventricular, intrahepatic, intracapsular, intrathecal, intracisternal, intraperitoneal, intranasal, intramyocardial, intra-coronary, aerosol, suppository, epicardial patch, oral administration, or by perfusion. For instance, therapeutic compositions for parenteral administration may be in the form of liquid solutions or suspensions; for oral administration, formulations may be in the form of tablets or capsules; and for intranasal formulations, in the form of powders, nasal drops, or aerosols. For instance, in some embodiments, a subject with a cardiac disease, such as acute myocardial infarction or heart failure, can be treated with a secretome-, extracellular vesicle-, and/or sEV-containing composition, produced from cardiomyocyte progenitor cells, cardiac progenitor cells, and/or cardiovascular progenitor cells, wherein the composition is administered intravenously.

The identified, selected, and/or formulated secretome-, extracellular vesicle-, and/or sEV-containing composition may be administered as a single dose. In other embodiments, multiple doses, spanning one or more doses per day, week, or month, may be administered to the subject. Single or repeated administration, including two, three, four, five or more administrations, may be made. The composition may also be administered continuously. Repeated or continuous administration may occur over a period of several hours (e.g., 1-2, 1-3, 1-6, 1-12, 1-18, or 1-24 hours), several days (e.g., 1-2, 1-3, 1-4, 1-5, 1-6 days, or 1-7 days) or several weeks (e.g., 1-2 weeks, 1-3 weeks, or 1-4 weeks), depending on the nature and/or severity of the condition being treated. If administration is repeated but not continuous, the time in between administrations may be hours (e.g., 4 hours, 6 hours, or 12 hours), days (e.g., 1 day, 2 days, 3 days, 4 days, 5 days, or 6 days), or weeks (e.g., 1 week, 2 weeks, 3 weeks, or 4 weeks). The time between administrations may be the same or they may differ. As an example, if symptoms worsen, or do not improve, the composition may be administered more frequently. Contrarily, if symptoms stabilize or diminish, the composition may be administered less frequently.

In some embodiments, a secretome-, extracellular vesicle-, and/or sEV-containing composition is administered in three doses, on or about two weeks apart, by intravenous administration. In some embodiments thereof, the composition may be diluted with, formulated with, and/or administered together with, a carrier, diluent, or suitable material (e.g., saline).

### EXPERIMENTAL

Non-limiting embodiments of the present invention are illustrated in the following Examples. Efforts have been made to ensure accuracy with respect to numbers used (e.g., amounts, concentrations, percent changes, and the like), but some experimental errors and deviations should be accounted for. It should be understood that these Examples are given by way of illustration only and are not intended to limit the scope of what the inventor regards as various embodiments of the present invention. Not all of the following steps set forth in each Example are required nor must the order of the steps in each Example be as presented.

### Example 1

### Reproducibility of H9c2 Viability Assay

To test the reproducibility of the H9c2 viability assay, the assay was performed essentially as described in El Harane et al. (Eur. Heart J., 2018; 39:1835-1847). In this assay, H9c2 cardiomyocytes are proliferative when culture media is rich in serum (e.g., cultured in H9c2 Complete Media), but cease to proliferate and loose viability when they are deprived of serum (e.g., cultured in H9c2 Poor Media). The capacity of extracellular vesicle (EV) preparations, including sEV, to promote H9c2 cardiomyocyte viability can therefore be determined by supplementing H9c2 Poor Media with EVs.

Briefly, H9c2 cells were thawed, and 4250 viable cells were seeded per well into the wells of a 96-well plate. After 24 hours of incubation in complete growth medium, the cells were again fed. After another 24 hours of incubation, the culture medium was removed, and either serum-containing medium (H9c2 Complete Media; for positive controls), or serum-free medium (H9c2 Poor Media; for test samples and negative controls), was added to appropriate wells (together with a nuclear-staining dye, to allow quantification of cell viability). sEV preparations (or mock sEV preparations) were then added to the appropriate wells, to determine the effect of the sEVs on H9c2 viability. The H9c2 cells were then cultured, and at several time points during the culturing, images were taken using an Incucyte (Essen BioSciences).

Using this assay, it was determined that different banks of H9c2 cells subject to serum-deprivation produced different cell viability results, even when using the same sEV preparations and culture conditions. Some sEV preparations were found to have positive effects on cell viability with a certain (high-passage) bank of H9c2 cells, but not with a different (low-passage) bank of H9c2 cells. This phenomenon was confirmed by manual cell-counting of viable cells (cells were manually counted using a hemocytometer after harvesting), and by using a ViCell XR cell viability analyzer (Beckman Coulter). An analysis of the different banks of H9c2 cells revealed that the H9c2 cells in the different banks exhibited different morphologies, depending on the passage number. The results suggested that there is too much H9c2 lot-to-lot, and passage-to-passage, variability in this H9c2 cell viability assay for this assay to become a standardized way of evaluating secretome or sEV potency. Furthermore, these rat cells may have limited applicability to the development and testing of human sEV products intended for human therapeutic development.

### Example 2

### Staurosporine Cardiomyocyte Viability Assay

In view of the results of the H9c2 cell viability assay in Example 1, an alternative cell viability assay was developed.

Specifically, human iCell Cardiomyocytes² (Fujifilm Cellular Dynamics, Inc., ref: CMC-100-012-001) were plated at 50,000 cells/well of a fibronectin-coated (5 μg/mL) 96-well plate in iCell Cardiomyocyte Plating Medium (Fujifilm Cellular Dynamics, Inc., ref: M1001), and cultured at 37° C. (atmospheric oxygen, 5% CO₂) for 4 hours. After this 4-hour incubation, the media was then exchanged for 100 μL (per well) of iCell Cardiomyocyte Maintenance Medium (iCMM, Fujifilm Cellular Dynamics, Inc., ref: M1003), and cells were cultured for up to 12 days, with full media exchanges every 2-3 days.

After a minimum of 4 days, cells were exposed to iCMM with NucSpot Live 650 dye (Biotium, ref: 40082; 0.125% final concentration) (this served as a viable cell (positive) control); or to iCMM with NucSpot Live 650 dye (0.125% final concentration), and staurosporine (Abcam, ref: ab146588) at a final in-well concentration of 2 μM (this also served as an apoptotic cell (negative) control). Dye, PBS and DMSO (0.325%) concentration, and final well volumes (120 μL), were equivalent in all wells. Cells were then cultured at 37° C. (atmospheric oxygen, 5% CO₂) for 4 hours.

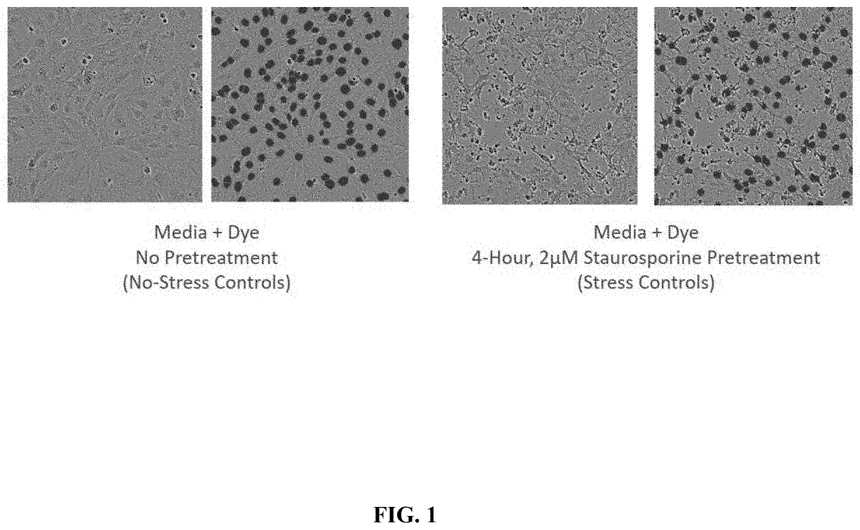

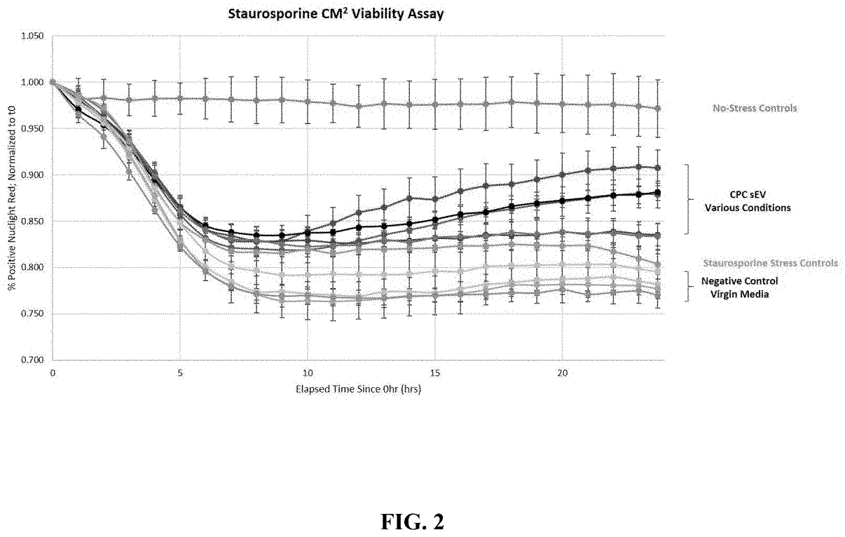

After this 4-hour incubation, the plate was imaged using an Incucyte (Essen BioSciences), the pre-incubation media was removed, and the wells were rinsed with 100 μL iCMM. Representative images for cells incubated with and without staurosporine are shown in FIG. 1. Cells were then fed with 120 μL (per well) of a mixture of iCMM containing NucSpot Live 650 dye (0.125% final concentration), DMSO (0.325% final concentration), and 15 μL DPB S (in a final volume of 120 μL); or with 120 μL (per well) of a mixture of iCMM containing NucSpot Live 650 dye (0.125% final concentration), DMSO (0.325% final concentration), and increasing concentrations of sEV or mock sEV preparations adjusted to 15 μL with DPBS. Cultures were then maintained at 37° C. (atmospheric oxygen, 5% CO₂). Wells were imaged in an Incucyte every hour for 24 hours, and nuclei counts were determined. Time course results were obtained by comparing % Positive NucSpot Live 650, Normalized to time 0 (T₀). FIG. 2 depicts this time course, showing that the sEV preparations, but not the mock sEV preparations, improved cardiomyocyte survival, indicating the functionality of the sEV preparation.

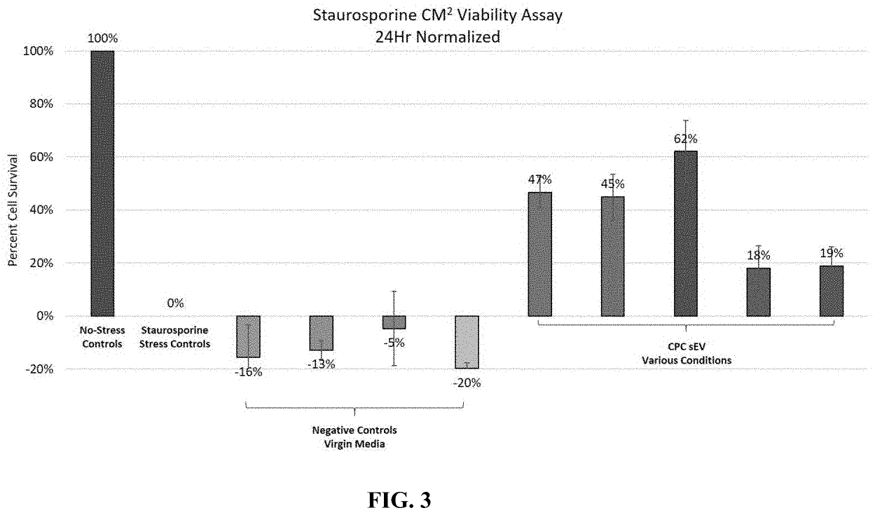

Since absolute numbers of cells per well may vary between assays, the data was treated to obtain comparable values, as follows. First, normalization was conducted by comparing, for each condition, the average number of cells at a specified time-point to the average number of cells at ime 0. Next, the normalized negative control value was subtracted from each condition, and the resulting value was compared to the difference between the positive and negative controls FIG. 3 depicts a histogram showing the results at the 24-hour time point of the staurosporine cardiomyocyte viability assay time course.

For the dosing of the sEVs, it was determined that protein concentration, RNA concentration, and sEV particle number, could vary considerably depending on the media, culture conditions, cell type, sEV isolation/enrichment technique, and donor. Accordingly, to ensure accurate dosing and accurate comparisons between different sEV samples, doses were calculated based on how many secreting cells generated the secretome. This was found to be a reliable measure in such an instance where the active components may be unknown; and non-active, co-isolated components, may vary considerably (see Example 4).

Additionally, as a confirmatory measure of cell viability, cell cultures in 96-well plates that had first been subjected to the above time course (Incucyte and bio-compatible dye) experiments were then subsequently analyzed for ATP content (i.e., at the end of the time course using the Incucyte and the bio-compatible dyes).

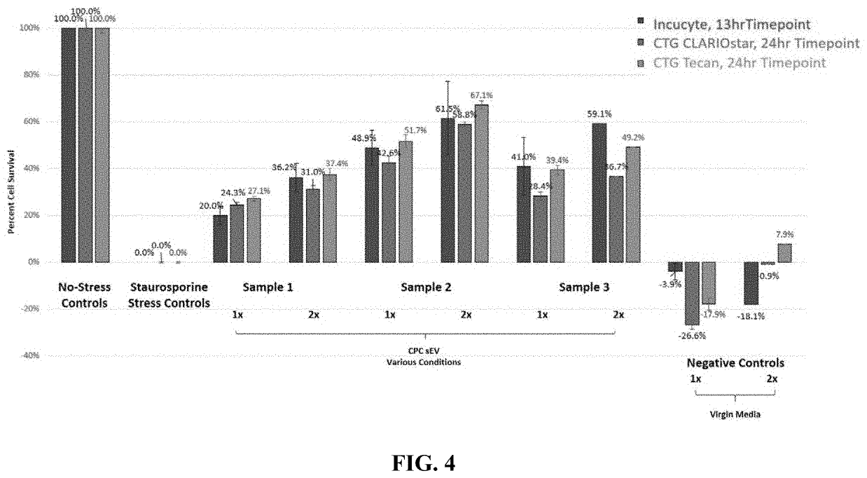

Specifically, the cells in each well were lyzed, and the ATP content therein was quantified using the CellTiter-Glo® Luminescent Cell Viability Assay (Promega) according to the manufacturer's directions. The resulting signal was analyzed using CLARIOStar® (BMG Labtech) and Tecan for Life Science® plate readers. The results are depicted in FIG. 4. FIG. 4 depicts the cell viability data obtained using the Incucyte at the 13 h timepoint, as well as the ATP quantification data obtained using the CellTiter-Glo® Luminescent Cell Viability Assay (Promega) with CLARIOStar® (BMG Labtech) and Tecan for Life Science® plate readers (at the 24 h timepoint, i.e., after the Incucyte time course had ended). As FIG. 4 shows, the results for the three datasets are strikingly similar to each other, confirming the use of the staurosporine assay with various detection methods and readouts.

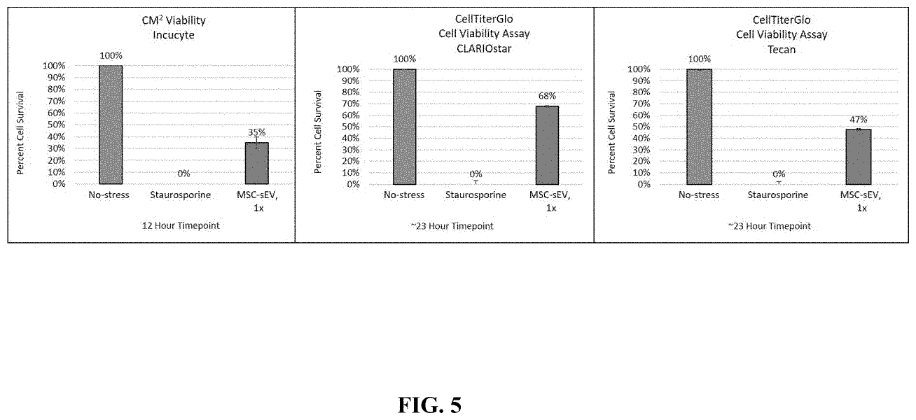

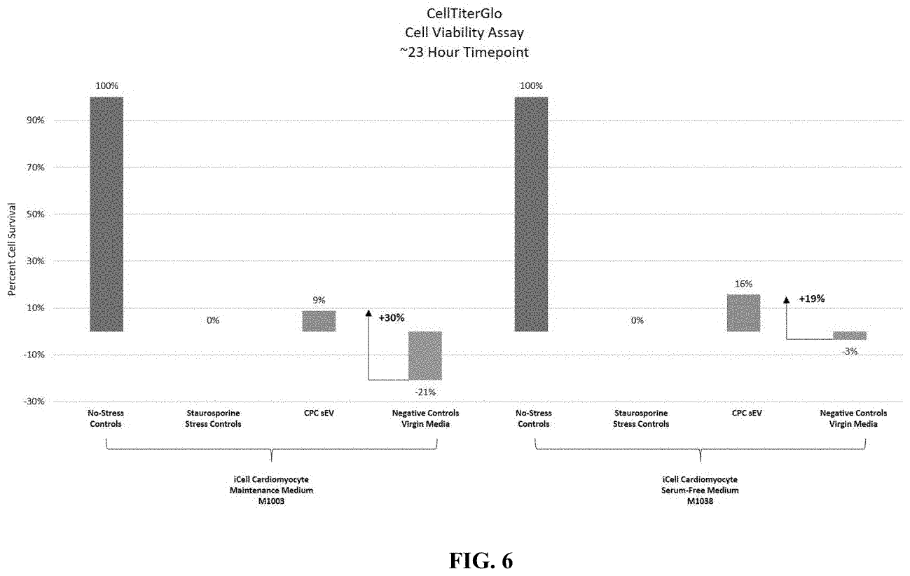

FIG. 5 depicts the results of an additional set of experiments in which cell viability data was obtained essentially as described above (using the Incucyte, with the data shown being from the 12 h timepoint), as well as ATP quantification data obtained using the CellTiter-Glo® Luminescent Cell Viability Assay (Promega) with CLARIOStar® (BMG Labtech) and Tecan for Life Science® plate readers (the data shown being from the 23 h timepoint, i.e., after the Incucyte time course had ended). FIG. 5 depicts the results of these experiments for a positive control (no staurosporine (“No-stress”)), a negative control with no sEV (“Staurosporine”), and for sEVs produced from mesenchymal stem cells (used at a 1× dose (“MSC-sEV, 1×”). As can be seen, the results are consistent with the results shown in FIG. 4. In a further set of experiments, human iCell Cardiomyocytes² were plated, cultured, and pre-treated with staurosporine (in iCMM medium, which contains serum) essentially as described above. However, following the staurosporine pre-treatment, cells were washed with either iCMM or iCell Cardiomyocyte Serum-Free Medium (iCSFM, Fujifilm Cellular Dynamics, Ref: M1038). Cells were then treated with PBS (vehicle), sEV, or MV in the same media type that the cells were washed with (i.e., either iCMM or iCSFM), and incubated at 37° C. (atmospheric oxygen, 5% CO₂). At the 23 h timepoint (i.e., 23 h after the staurosporine pretreatment commenced), the cells in each well were lyzed, and the ATP content therein was quantified using the CellTiter-Glo® Luminescent Cell Viability Assay (Promega) according to the manufacturer's directions. The resulting signal was analyzed using a Tecan for Life Science® plate reader. The results are depicted in FIG. 6, which demonstrates that the EV treatment phase, at least, may be conducted in the absence of serum, while providing comparable results.

### Example 3

### Comparison of Staurosporine Cardiomyocyte Viability Assay to HUVEC Scratch Assay

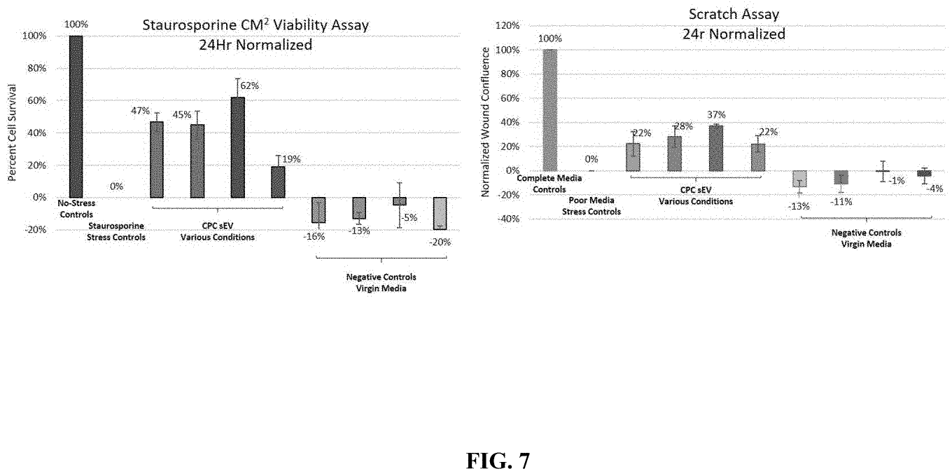

To confirm the validity of the staurosporine cardiomyocyte viability assay described in Example 2, a HUVEC scratch wound healing assay (developed by Essen BioSciences, for the Incucyte) was conducted on the same sEV and mock sEV preparations. Briefly, HUVEC cells were expanded using HUVEC Complete Media: Endothelial Cell Basal Media (PromoCell, Ref: C-22210), supplemented with the Endothelial Cell Growth Medium Supplement Pack (PromoCell, Ref: C-39210). After expansion, the cells were cryopreserved in CS10 (Cryostore, ref: 210102) at 1-2×10⁶ cells per aliquot (enough for between a half to a full 96-well plate). Two days prior to assay, HUVEC aliquots were thawed, and plated onto ImageLock 96-well plates (EssenBio, Ref: 4379) at 10,000 cells/well, and grown in HUVEC Complete media for two days. Cultures were then maintained at 37° C. (atmospheric oxygen, 5% CO₂) throughout the maintenance and assay process. Wells were scratched using a Wound Maker (EssenBio, Ref: 4493) according to the manufacturer's directions, and cells were then rinsed with Endothelial Cell Basal Media and cultured overnight (either in HUVEC Complete Media alone, as a positive control; in Endothelial Cell Basal Media alone, as a negative control; or in Endothelial Cell Basal Media supplemented with sEV or mock sEV preparations). Using an Incucyte with the Scratch Wound Healing Module, plates were imaged every three hours for a total of 18 hours. Wound closure was determined using the manufacturer's software, and values were baseline (negative control) subtracted, and normalized to the positive control. FIG. 7 provides a comparison between the results of the staurosporine cardiomyocyte viability assay and the HUVEC scratch wound healing assay, showing that the staurosporine cardiomyocyte viability assay provides consistent results comparable to the HUVEC scratch assay, but here on a potentially more relevant cell type to cardiac disease, i.e., cardiomyocytes.

### Example 4

### Dosing sEV by Particle Number as Compared to Cell Number

As discussed in Example 2, a dosing strategy was needed that would ensure accurate comparisons between different sEV samples. Initially, sEV doses were calculated based on the number of particles per sEV sample, with a “1×” dosing volume being equivalent to a target of 1 billion particles as identified by the NanoSight. The Mock sEV dosing strategy was to match dosing volumes with sEV sample “1×” dosing volumes. If more than one sEV sample was present, the Mock sEV was matched to whichever “1×” sEV sample volume was largest.

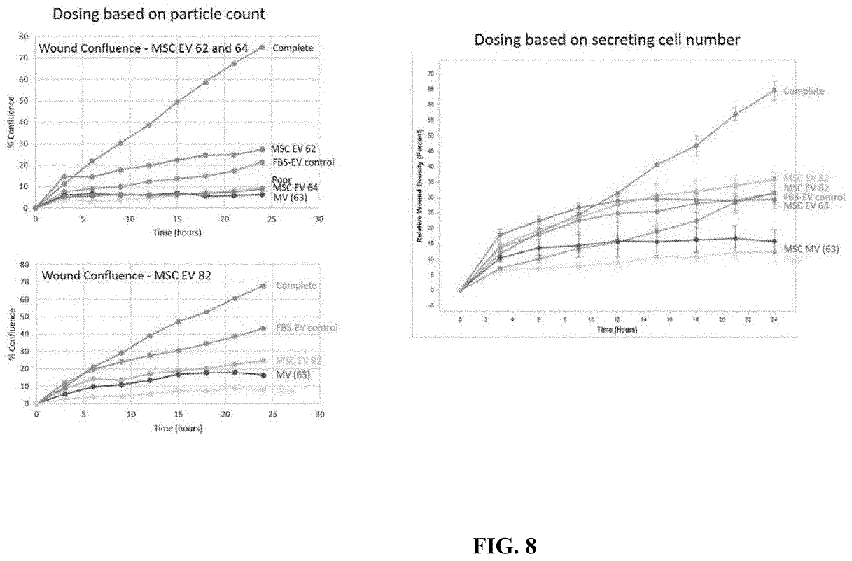

Variability in sEV sample functionality was noted when 1 billion particles from different MSC donor lots were compared to each other in a HUVEC scratch wound healing assay. These MSC donors were cultured, harvested, and had sEV samples prepared under similar conditions. To investigate the functionality differences between these lots, an alternative dosing strategy was considered. In this new strategy, doses were calculated based on how many secreting cells generated the sEV secretome. When assessed in the HUVEC scratch wound healing assay, dosing sEV samples from each MSC lot at “1×” volumes equal to the secretome secretions of 1.85×10⁵ cells resulted in more similar functionality results as compared to dosing by particle number (see FIG. 8).

### Example 5

### Considerations Taken When Matching Mock sEV to sEV Preparations

As mentioned in Example 4, sEV sample dosing was originally based on particle count, and Mock sEV dosing relied on a “1×” dose volume-to-volume matching strategy with sEV samples. However, when a new sEV sample dosing strategy was adopted that based doses on how many secreting cells generated the sEV secretome, a new Mock sEV dosing strategy was also needed. For this strategy, the volume of mock sEV is matched to an sEV sample as follows:

For the matched sEV sample, the ratio of final sEV preparation volume to conditioned media inputted into the isolation/enrichment procedure is calculated (“μL sEV/mL MC”). Similarly, for mock sEV samples, the volume of the final preparation is divided by the volume of virgin media subjected to the isolation/enrichment method (“μL, MV/mL MV”). When the sEV sample is used in the staurosporine assay or other in vitro or in vivo assay, the “MC media equivalents” of the dose is calculated (“MC media equivalents”=volume of sEV dose in the assay/“μL sEV/mL MC”). The volume of MV needed as a control in the assay can then be calculated to match the MC media equivalents and therefore control for any effect that might be coming from contaminating components from the media itself (volume MV to use as a control=“uL MV/mL MV”*“MC media equivalents”).

### Example 6

### Staurosporine Cardiomyocyte Attachment/Growth/Number Assay (Electrical Impedance)

In the staurosporine cardiomyocyte viability assay experiments described in Example 2, cell viability was determined (by live cell imaging using an Incucyte and biocompatible dyes; and by measuring ATP content). To demonstrate that the staurosporine assay described in Example 2 can be used with alternate detection methods or readouts to determine the functionality of an sEV preparation, the staurosporine assay described in Example 2 was performed using cell growth/adhesion/number as a measure of the functionality of an sEV preparation.

Specifically, human iCell Cardiomyocytes² (Fujifilm Cellular Dynamics, Inc., ref: CMC-100-012-001) were first seeded onto fibronectin-coated wells of a culture plate equipped with electrodes (CytoView Z-Plate, Axion Biosystems®) in iCell Cardiomyocyte Maintenance Medium (iCMM, Fujifilm Cellular Dynamics, Inc., ref: M1003) at 37° C. (atmospheric oxygen, 5% CO₂), and allowed to adhere and form a monolayer (“0 h”).

After 126 h, at which time the seeded cells had adhered and formed a confluent monolayer (as determined by a plateau in impedance values, after an increase from zero at 0 h), the cells were washed and then exposed to a pre-incubation medium of iCMM (this served as a viable cell (positive) control); or iCMM containing staurosporine (Abcam, ref: ab146588) at a final in-well concentration of 2 μM (this also served as an apoptotic cell (negative) control). Cells were then cultured at 37° C. (atmospheric oxygen, 5% CO₂) for 4 hours. Impedance was continually measured during the incubation using an Axion BioSystems® Maestro Z device.

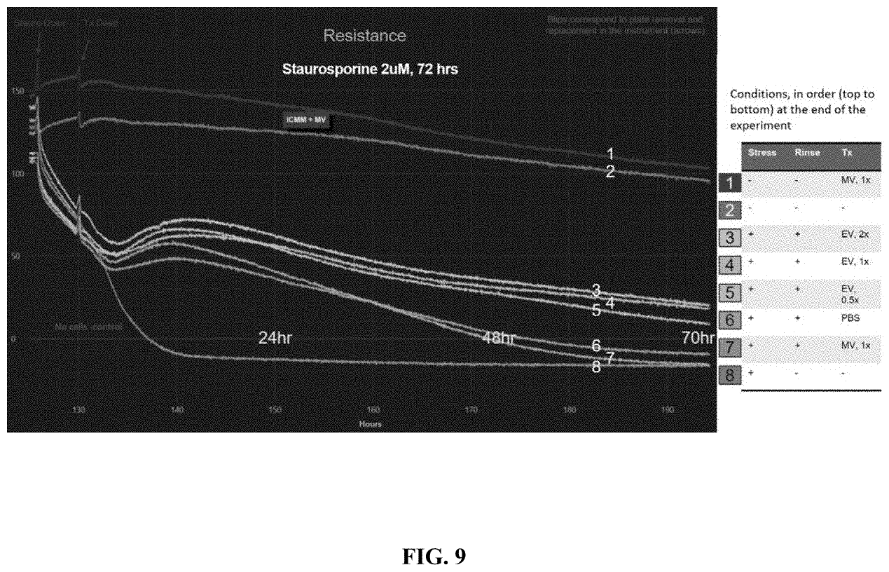

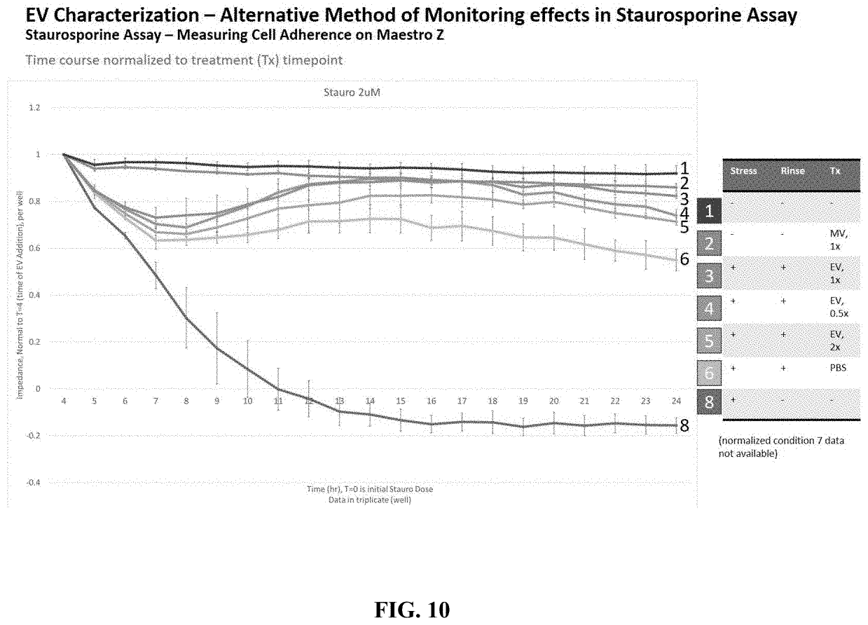

After this 4-hour incubation, the pre-incubation media was removed (except in a control sample in which staurosporine was present during the subsequent incubation, see Condition 8 in FIG. 9), and the wells were rinsed with iCMM. Different wells of cells were then either fed with iCMM (see Conditions 2 and 8 in FIG. 9), or with iCMM supplemented with: varying concentrations of sEV (see Conditions 3-5 in FIG. 9); mock sEV preparations (virgin media controls, see Conditions 1 and 7 in FIG. 9); or PBS (see Condition 6 in FIG. 9). Cultures were then maintained at 37° C. (atmospheric oxygen, 5% CO₂) for 72 h, and impedance was again continually measured. FIG. 9 depicts this time course, showing that the sEV preparations (see Conditions 3-5 in FIG. 9), but not the mock sEV preparation (see Condition 7 in FIG. 9), improved cardiomyocyte attachment after staurosporine treatment, as compared to the negative (staurosporine-treated) controls (see Conditions 6 and 8 in FIG. 9). The positive controls are shown by Conditions 1 and 2 in FIG. 9. These results, like the results of the cardiomyocyte viability assay experiments described in Example 2, indicated the functionality of the sEV preparation. FIG. 10 shows the results of the experiments normalized to the treatment (“Tx”) timepoint.

1. A method for analyzing an activity of a secretome, said method comprising:
(a) contacting a culture of target cells with a pre-treatment medium, and culturing said target cells in said pre-treatment medium under at least one stress-inducing condition;
(b) administering a secretome to the cell culture, and culturing said target cells in the presence of the secretome; and
(c) measuring at least one property of the cultured cells one or more times during the culturing of step (b).

2. The method of claim 1, wherein said method further comprises removing the pre-treatment medium from the cultured cells before step (b).

3. The method of claim 2, wherein the target cells are cultured under the at least one stress-inducing condition before the administration of the secretome to the cell culture, and wherein the culturing of the target cells in step (b) is performed in the absence of the at least one stress-inducing condition.

4. A method for analyzing an activity of a secretome, said method comprising:
(a) contacting a culture of target cells with a pre-treatment medium, and culturing said target cells in said pre-treatment medium;
(b) administering a secretome to the cell culture, and optionally culturing said target cells in the presence of the secretome;
(c) culturing the target cells under at least one stress-inducing condition; and
(d) measuring at least one property of the cultured cells one or more times during the culturing of step (c).

5. The method of claim 4, wherein said target cells are cultured in the presence of the secretome before the culturing of step (c).

6. The method of claim 5, wherein said method further comprises removing the secretome from the cultured cells before step (c).

7. The method of claim 4, wherein the stress-inducing condition is culturing in the presence of a cellular stress agent, wherein the cellular stress agent is co-administered with the secretome, and wherein said target cells are cultured in the presence of the secretome and the cellular stress agent.

8. The method of any one of claim 1 wherein the at least one stress-inducing culture condition is culturing in the presence of a cellular stress agent.

9. The method of claim 7, wherein said cellular stress agent is a chemotherapeutic agent and/or an apoptosis-inducing agent.

10. The method of claim 8, wherein said cellular stress agent is a chemotherapeutic agent and/or an apoptosis-inducing agent.

11. The method of claim 9, wherein said apoptosis-inducing agent is selected from an indolocarbazole, an indolo(2,3-a)pyrrole(3,4-c)carbazole or a derivative thereof, staurosporine or a derivative thereof, and/or doxorubicin or a derivative thereof.

12. The method of claim 10, wherein said apoptosis-inducing agent is selected from an indolocarbazole, an indolo(2,3-a)pyrrole(3,4-c)carbazole or a derivative thereof, staurosporine or a derivative thereof, and/or doxorubicin or a derivative thereof.

13. The method of claim 1, wherein the at least one property that is measured is selected from the group consisting of cell viability, hypertrophy, cell health, cell adhesion, cell physiology, ATP content, cell number, and cell morphology.

14. The method of claim 1, wherein the at least one property is selected from cell viability, cell adhesion, cell number, cell morphology, cell growth, and/or ATP content.

15. The method of claim 1, wherein said secretome is isolated from a culture of one or more cells selected from totipotent progenitor cells, multipotent progenitor cells, and end-stage differentiated cells.

16. The method of claim 1, wherein said secretome comprises a small extracellular vesicle-enriched fraction (sEV) isolated from a cell culture.

17. A method for analyzing an activity of a small molecule therapeutic, said method comprising:
(a) contacting a culture of target cells with a pre-treatment medium, and culturing said target cells in said pre-treatment medium in the presence of the small molecule or chemotherapy agent;
(b) administering a secretome to the cell culture, and culturing said target cells in the presence of the secretome; and
(c) measuring at least one property of the cultured cells one or more times during the culturing of step (b).
